(19) United States
(12) Patent Application Publication
(10) Pub. No.: US 2025/0340651 A1
(43) Pub. Date: Nov. 6, 2025

(54) ANTI-MICA/B ANTIBODIES WITH ENHANCED EFFECTOR FUNCTION AND METHODS OF USE

(71) Applicant: CULLINAN MICA CORPORATION, Cambridge, MA (US)

(72) Inventors: Jennifer S. Michaelson, Cambridge, MA (US); Naveen K. Mehta, Cambridge, MA (US); Patrick A. Baeuerle, Cambridge, MA (US); Kerry A. Whalen, Cambridge, MA (US)

(21) Appl. No.: 18/998,692

(22) PCT Filed: Jul. 27, 2023
(86) PCT No.: PCT/US2023/071131

§ 371 Date: Jan. 27, 2025

(51) Int. Cl. C07K 16/28; A61P 35/00

(52) U.S. Cl. CPC C07K 16/2833; A61P 35/00; C07K 2317/24; C07K 2317/31

(57) ABSTRACT

Provided herein are antibodies or antigen-binding fragments thereof that specifically bind to MICA/B having heavy chain, light chain, variable heavy chain domains (VH), variable light chain domains (VL), and complementarity determining regions (CDRs) disclosed herein, as well as methods and uses thereof. In certain aspects, the antibodies or an antigen-binding fragment thereof comprise one or more amino acid modifications to enhance effector function compared to the unmodified monoclonal antibody or antigen-binding fragment thereof; and/or lacks fucosylation or comprises reduced fucosylation.

### CROSS-REFERENCE TO RELATED APPLICATIONS

The present patent application claims the priority benefit of U.S. Provisional Patent Application Ser. No. 63/393,188, filed Jul. 28, 2022, the content of which is hereby incorporated by reference in its entirety into this disclosure.

### REFERENCE TO AN ELECTRONIC SEQUENCE LISTING

The contents of the electronic sequence listing (67253WO01 Sequence Listing.xml; Size: 42 kilobytes; and Date of Creation: 2023-07-27) is herein incorporated by reference in its entirety.

### SUMMARY

Disclosed herein, are monoclonal antibodies that specifically bind to MICA/B and thereby modulating an immune response against disease cells.

Disclosed herein, in certain embodiments, are monoclonal antibodies or an antigen-binding fragments thereof, comprising a light chain comprising an amino acid sequence at least 80% identical to an amino acid sequence set forth as SEQ ID NO: 21, SEQ ID NO: 23, or SEQ ID NO: 24. In some embodiments, the monoclonal antibodies or an antigen-binding fragments thereof further comprises a heavy chain comprising an amino acid sequence at least 80% identical to an amino acid sequence set forth as SEQ ID NO: 22, SEQ ID NO: 25, or SEQ ID NO: 26; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain variable domain (VL) comprising an amino acid sequence at least 80% identical to an amino acid sequence set forth as SEQ ID NO: 7, SEQ ID NO: 15, or SEQ ID NO: 19. Disclosed herein, in certain embodiments, are monoclonal antibodies or an antigen-binding fragments thereof, comprising a light chain variable domain (VL) comprising an amino acid sequence at least 90% identical to an amino acid sequence set forth as SEQ ID NO: 7, SEQ ID NO: 15, or SEQ ID NO: 19. Disclosed herein, in certain embodiments, are monoclonal antibodies or an antigen-binding fragments thereof, comprising a light chain variable domain (VL) comprising an amino acid sequence at least 95% identical to an amino acid sequence set forth as SEQ ID NO: 7, SEQ ID NO: 15, or SEQ ID NO: 19. Disclosed herein, in certain embodiments, are monoclonal antibodies or an antigen-binding fragments thereof, comprising a light chain variable domain (VL) comprising an amino acid sequence at least 99% identical to an amino acid sequence set forth as SEQ ID NO: 7, SEQ ID NO: 15, or SEQ ID NO: 19; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain variable domain (VL) comprising an amino acid sequence 100% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, or SEQ ID NO: 20. In some instances, the monoclonal antibody or antigen-binding fragment thereof comprises a heavy chain variable domain (VH) comprising an amino acid sequence at least 90% identical to an amino acid sequence set forth as SEQ ID NO: 8, SEQ ID NO: 16, or SEQ ID NO: 20. In some instances, the monoclonal antibody or antigen-binding fragment thereof comprises a heavy chain variable domain (VH) comprising an amino acid sequence at least 95% identical to an amino acid sequence set forth as SEQ ID NO: 8, SEQ ID NO: 16, or SEQ ID NO: 20. In some instances, the monoclonal antibody or antigen-binding fragment thereof comprises a heavy chain variable domain (VH) comprising an amino acid sequence at least 99% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In some instances, the monoclonal antibody or antigen-binding fragment thereof comprises a heavy chain variable domain (VH) comprising an amino acid sequence 100% identical to an amino acid sequence set forth as SEQ ID NO: 8, SEQ ID NO: 16, or SEQ ID NO: 20. In some instances, the monoclonal antibody or antigen-binding fragment thereof comprises alight chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or TgM. In some instances, the monoclonal antibody or antigen-binding fragment thereof is humanized or chimeric.

Disclosed herein, in certain embodiments, are monoclonal antibodies or an antigen-binding fragments thereof, comprising a heavy chain variable domain (VH) comprising an amino acid sequence at least 80% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain variable domain (VH) comprising an amino acid sequence at least 90% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain variable domain (VH) comprising an amino acid sequence at least 95% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain variable domain (VH) comprising an amino acid sequence at least 99% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain variable domain (VH) comprising an amino acid sequence 100% identical to an amino acid sequence set forth as SEQ ID NO: 8, SEQ ID NO: 16, or SEQ ID NO: 20; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19. In some instances, the monoclonal antibody or antigen-binding fragment thereof comprises a light chain variable domain (VL) comprising an amino acid sequence at least 90% identical to an amino acid sequence set forth as SEQ ID NO: 7, SEQ ID NO: 15, or SEQ ID NO: 19. In some instances, the monoclonal antibody or antigen-binding fragment thereof comprises a light chain variable domain (VL) comprising an amino acid sequence at least 95% identical to an amino acid sequence set forth as SEQ ID NO: 7, SEQ ID NO: 15, or SEQ ID NO: 19. In some instances, the monoclonal antibody or antigen-binding fragment thereof comprises a light chain variable domain (VL) comprising an amino acid sequence at least 99% identical to an amino acid sequence set forth as SEQ ID NO: 7, SEQ ID NO: 15, or SEQ ID NO: 19. In some instances, the monoclonal antibody or antigen-binding fragment thereof comprises a light chain variable domain (VL) comprising an amino acid sequence 100% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to SEQ ID NO: 8 and a light chain variable domain (VL) comprising an amino acid sequence at least 80% identical to SEQ ID NO: 7. In some instances, the monoclonal antibody or antigen-binding fragment thereof comprises a heavy chain variable domain (VH) comprising an amino acid sequence at least 80% identical to SEQ ID NO: 16 and a light chain variable domain (VL) comprising an amino acid sequence at least 80% identical to SEQ ID NO: 15. In some instances, the monoclonal antibody or antigen-binding fragment thereof comprises a heavy chain variable domain (VH) comprising an amino acid sequence at least 80% identical to SEQ ID NO: 20 and a light chain variable domain (VL) comprising an amino acid sequence at least 80% identical to SEQ ID NO: 19. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or antigen-binding fragment thereof is humanized or chimeric.

Disclosed herein, in certain embodiments, are monoclonal antibodies or an antigen-binding fragments thereof, comprising a light chain complementarity determining region (CDR) having an amino acid sequence at least 80% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain complementarity determining region (CDR) having an amino acid sequence at least 90% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain complementarity determining region (CDR) having an amino acid sequence at least 95% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain complementarity determining region (CDR) having an amino acid sequence at least 99% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a light chain complementarity determining region (CDR) having an amino acid sequence 100% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In some instances, the monoclonal antibody or antigen-binding fragment thereof comprises a heavy chain complementarity determining region (CDR) having an amino acid sequence at least 80% identical to at least one of the amino acid sequences set forth as SEQ ID NOS: 4-6, 12-14, and 18. In some instances, the monoclonal antibody or antigen-binding fragment thereof comprises a heavy chain complementarity determining region (CDR) having an amino acid sequence at least 90% identical to at least one of the amino acid sequences set forth as SEQ ID NOS: 4-6, 12-14, and 18. In some instances, the monoclonal antibody or antigen-binding fragment thereof comprises a heavy chain complementarity determining region (CDR) having an amino acid sequence at least 95% identical to at least one of the amino acid sequences set forth as SEQ ID NOS: 4-6, 12-14, and 18. In some instances, the monoclonal antibody or antigen-binding fragment thereof comprises a heavy chain complementarity determining region (CDR) having an amino acid sequence at least 99% identical to at least one of the amino acid sequences set forth as SEQ ID NOS: 4-6, 12-14, and 18. In some instances, the monoclonal antibody or antigen-binding fragment thereof comprises a heavy chain complementarity determining region (CDR) having an amino acid sequence 100% identical to at least one of the amino acid sequences set forth as SEQ ID NOS: 4-6, 12-14, and 18. In some instances, the monoclonal antibody or antigen-binding fragment thereof comprises a light chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NOS: SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5. SEQ ID NO: 13, or SEQ ID NO: 18, and a heavy chain complementarity determining region 3 (CDR3) having an amino acid sequence at least 80% identical to one of SEQ ID NO: 6, or SEQ ID NO: 14. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 1 (CDR1) having an amino acid sequence at least 80% identical to SEQ ID NO: 17, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO: 20. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15; and a heavy chain variable domain (VH) comprising an amino acid sequence at least 80% identical to an amino acid sequence set forth as SEQ ID NO: 8, or SEQ ID NO: 16. In some instances, the monoclonal antibody or antigen-binding fragment thereof comprises a heavy chain variable domain (VH) comprising an amino acid sequence at least 80% identical to an amino acid sequence set forth as SEQ ID NO: 8, or SEQ ID NO: 16; and a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or antigen-binding fragment thereof is humanized or chimeric.

Disclosed herein, in certain embodiments, are monoclonal antibodies or an antigen-binding fragments thereof, comprising a heavy chain complementarity determining region (CDR) having an amino acid sequence at least 80% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain complementarity determining region (CDR) having an amino acid sequence at least 90% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain complementarity determining region (CDR) having an amino acid sequence at least 95% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain complementarity determining region (CDR) having an amino acid sequence at least 99% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof, comprising a heavy chain complementarity determining region (CDR) having an amino acid sequence 100% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

In some instances, the monoclonal antibody or antigen-binding fragment comprises a light chain complementarity determining region (CDR) having an amino acid sequence at least 80% identical to at least one of the amino acid sequences set forth as SEQ ID NOS: 1-3, 9-11, and 17. In some instances, the monoclonal antibody or antigen-binding fragment comprises a light chain complementarity determining region (CDR) having an amino acid sequence at least 90% identical to at least one of the amino acid sequences set forth as SEQ ID NOS: 1-3, 9-11, and 17. In some instances, the monoclonal antibody or antigen-binding fragment comprises a light chain complementarity determining region (CDR) having an amino acid sequence at least 95% identical to at least one of the amino acid sequences set forth as SEQ ID NOS: 1-3, 9-11, and 17. In some instances, the monoclonal antibody or antigen-binding fragment comprises a light chain complementarity determining region (CDR) having an amino acid sequence at least 99% identical to at least one of the amino acid sequences set forth as SEQ ID NOS: 1-3, 9-11, and 17. In some instances, the monoclonal antibody or antigen-binding fragment comprises a light chain complementarity determining region (CDR) having an amino acid sequence 100% identical to at least one of the amino acid sequences set forth as SEQ ID NOS: 1-3, 9-11, and 17. In some instances, the monoclonal antibody or antigen-binding fragment thereof comprises a heavy chain complementarity determining region 1 (CDR1) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5, SEQ ID NO: 13, or SEQ ID NO: 18, a heavy chain complementarity determining region 3 (CDR3) having an amino acid sequence at least 80% identical to one of SEQ ID NO: 6, or SEQ ID NO: 14, a light chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NO: 2, or SEQ ID NO: 10, and a light chain complementarity determining region 3 (CDR3) having an amino acid sequence at least 80% identical to one of SEQ ID NO: 3, or SEQ ID NO: 11. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 1 (CDR1) having an amino acid sequence at least 80% identical to SEQ ID NO: 17, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO:20. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO:20; and a light chain variable domain (VL) comprising an amino acid sequence at least 80% identical to an amino acid sequence set forth as SEQ ID NO: 7, SEQ ID NO: 15, or SEQ ID NO:19. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15; and a heavy chain variable domain (VH) comprising an amino acid sequence at least 80% identical to an amino acid sequence set forth as SEQ ID NO: 8, or SEQ ID NO: 16. In some instances, the monoclonal antibody or antigen-binding fragment thereof comprises a heavy chain variable domain (VH) comprising an amino acid sequence at least 80% identical to an amino acid sequence set forth as SEQ ID NO: 8 and a light chain variable domain (VL) comprising an amino acid sequence at least 80% identical to an amino acid sequence set forth as SEQ ID NO: 7. In some instances, the monoclonal antibody or antigen-binding fragment thereof comprises a heavy chain variable domain (VH) comprising an amino acid sequence at least 80% identical to an amino acid sequence set forth as SEQ ID NO: 16; and a light chain variable domain (VL) comprising an amino acid sequence at least 80% identical to an amino acid sequence set forth as SEQ ID NO: 15. In some instances, the monoclonal antibody or antigen-binding fragment thereof comprises a heavy chain variable domain (VH) comprising an amino acid sequence at least 80% identical to an amino acid sequence set forth as SEQ ID NO: 20; and a light chain variable domain (VL) comprising an amino acid sequence at least 80% identical to an amino acid sequence set forth as SEQ ID NO: 19. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or antigen-binding fragment thereof is humanized or chimeric.

Disclosed herein, in certain embodiments, are pharmaceutical compositions comprising: a monoclonal antibody or an antigen-binding fragment thereof according to any one of the disclosures herein; and a pharmaceutically acceptable carrier or excipient; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are methods of treating cancer in an individual in need thereof, comprising administering to the individual an effective amount of a monoclonal antibody or an antigen-binding fragment thereof comprising a light chain complementarity determining region (CDR) having an amino acid sequence at least 80% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are methods of treating cancer in an individual in need thereof, comprising administering to the individual an effective amount of a monoclonal antibody or an antigen-binding fragment thereof comprising a heavy chain complementarity determining region (CDR) having an amino acid sequence at least 80% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In some instances, the monoclonal antibody or antigen-binding fragment thereof comprises (a) a light chain complementarity determining region (CDR) having an amino acid sequence at least 80% identical to at least one of the amino acid sequences set forth as SEQ ID NOS: 1-3, 9-11, and 17; and (b) a heavy chain complementarity determining region (CDR) having an amino acid sequence at least 80% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 90% identical to at least one of the amino acid sequences set forth as SEQ ID NOS: 1-3, 9-11, and 17; and (b) a heavy chain complementarity determining region (CDR) having an amino acid sequence at least 90% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 95% identical to at least one of the amino acid sequences set forth as SEQ ID NOS: 1-3, 9-11, and 17; and (b) a heavy chain complementarity determining region (CDR) having an amino acid sequence at least 95% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 99% identical to at least one of the amino acid sequences set forth as SEQ ID NOS: 1-3, 9-11, and 17; and (b) a heavy chain complementarity determining region (CDR) having an amino acid sequence at least 99% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence 100% identical to at least one of the amino acid sequences set forth as SEQ ID NOS: 1-3, 9-11, and 17; and (b) a heavy chain complementarity determining region (CDR) having an amino acid sequence 100% identical to at least one of the amino acid sequences set forth as SEQ ID NOS: 4-6, 12-14, and 18. In some instances, the monoclonal antibody or antigen-binding fragment thereof comprises a light chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NOS: SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5, SEQ ID NO: 13, or SEQ ID NO: 18, and a heavy chain complementarity determining region 3 (CDR3) having an amino acid sequence at least 80% identical to one of SEQ ID NO: 6, or SEQ ID NO: 14. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDRT) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 17 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO: 20. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19; and a heavy chain variable domain (VH) comprising an amino acid sequence at least 80% identical to an amino acid sequence set forth as SEQ ID NO: 8, SEQ ID NO: 16, or SEQ ID NO: 20. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or TgM. In some instances, the monoclonal antibody or antigen-binding fragment thereof is humanized or chimeric. In some instances, the monoclonal antibody or antigen-binding fragment thereof reduces level of soluble MICA protein, soluble MICB protein, or both. In some instances, the monoclonal antibody or antigen-binding fragment thereof reduces shedding of soluble MICA protein, soluble MICB protein, or both. In some instances, the monoclonal antibody or antigen-binding fragment thereof inhibits shedding of soluble MICA protein, soluble MICB protein, or both.

Disclosed herein, in certain embodiments, are methods of treating cancer in an individual in need thereof, comprising administering to the individual an effective amount of a monoclonal antibody or an antigen-binding fragment thereof comprising a light chain complementarity determining region (CDR) having an amino acid sequence at least 80% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

Disclosed herein, in certain embodiments, are methods of treating cancer in an individual in need thereof, comprising administering to the individual an effective amount of a monoclonal antibody or an antigen-binding fragment thereof comprising a heavy chain complementarity determining region (CDR) having an amino acid sequence at least 80% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In some instances, the monoclonal antibody or antigen-binding fragment thereof comprises (a) a light chain complementarity determining region (CDR) having an amino acid sequence at least 80% identical to at least one of the amino acid sequences set forth as SEQ ID NOS: 1-3, 9-11, and 17; and (b) a heavy chain complementarity determining region (CDR) having an amino acid sequence at least 80% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 90% identical to at least one of the amino acid sequences set forth as SEQ ID NOS: 1-3, 9-11, and 17; and (b) a heavy chain complementarity determining region (CDR) having an amino acid sequence at least 90% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 95% identical to at least one of the amino acid sequences set forth as SEQ ID NOS: 1-3, 9-11, and 17; and (b) a heavy chain complementarity determining region (CDR) having an amino acid sequence at least 95% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 99% identical to at least one of the amino acid sequences set forth as SEQ ID NOS: 1-3, 9-11, and 17; and (b) a heavy chain complementarity determining region (CDR) having an amino acid sequence at least 99% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence 100% identical to at least one of the amino acid sequences set forth as SEQ ID NOS: 1-3, 9-11, and 17; and (b) a heavy chain complementarity determining region (CDR) having an amino acid sequence 100% identical to at least one of the amino acid sequences set forth as SEQ ID NOS: 4-6, 12-14, and 18. In some instances, the monoclonal antibody or antigen-binding fragment thereof comprises a light chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NOS: SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5, SEQ ID NO: 13, or SEQ ID NO: 18, and a heavy chain complementarity determining region 3 (CDR3) having an amino acid sequence at least 80% identical to one of SEQ ID NO: 6, or SEQ ID NO: 14. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 17 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO: 20. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19; and a heavy chain variable domain (VH) comprising an amino acid sequence at least 80% identical to an amino acid sequence set forth as SEQ ID NO: 8, SEQ ID NO: 16, or SEQ ID NO: 20. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof specifically binds to the alpha 3 domain of MICB. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or fragment thereof is humanized or chimeric. In some instances, the monoclonal antibody or antigen binding fragment thereof reduces level of soluble MICA protein, soluble MICB protein, or both. In some instances, the monoclonal antibody or antigen binding fragment thereof reduces shedding of soluble MICA protein, soluble MICB protein, or both. In some instances, the monoclonal antibody or antigen binding fragment thereof inhibits shedding of soluble MICA protein, soluble MICB protein, or both.

Disclosed herein, in certain embodiments, are methods of reducing level of soluble MICA protein, soluble MICB protein, or both in an individual in need thereof, comprising administering to the individual an effective amount of a monoclonal antibody or an antigen-binding fragment thereof comprising a light chain complementarity determining region (CDR) having an amino acid sequence at least 80% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY.

Disclosed herein, in certain embodiments, are methods of reducing level of soluble MICA protein, soluble MICB protein, or both in an individual in need thereof, comprising administering to the individual an effective amount of a monoclonal antibody or an antigen-binding fragment thereof comprising a heavy chain complementarity determining region (CDR) having an amino acid sequence at least 80% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY. In some instances, the monoclonal antibody or antigen-binding fragment thereof comprises (a) a light chain complementarity determining region (CDR) having an amino acid sequence at least 80% identical to at least one of the amino acid sequences set forth as SEQ ID NOS: 1-3, 9-11, and 17; and (b) a heavy chain complementarity determining region (CDR) having an amino acid sequence at least 80% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 90% identical to at least one of the amino acid sequences set forth as SEQ ID NOS: 1-3, 9-11, and 17; and (b) a heavy chain complementarity determining region (CDR) having an amino acid sequence at least 90% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 95% identical to at least one of the amino acid sequences set forth as SEQ ID NOS: 1-3, 9-11, and 17; and (b) a heavy chain complementarity determining region (CDR) having an amino acid sequence at least 95% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 99% identical to at least one of the amino acid sequences set forth as SEQ ID NOS: 1-3, 9-11, and 17; and (b) a heavy chain complementarity determining region (CDR) having an amino acid sequence at least 99% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence 100% identical to at least one of the amino acid sequences set forth as SEQ ID NOS: 1-3. 9-11, and 17, and (b) a heavy chain complementarity determining region (CDR) having an amino acid sequence 100% identical to at least one of the amino acid sequences set forth as SEQ ID NOS: 4-6, 12-14, and 18. In some instances, the monoclonal antibody or antigen-binding fragment thereof comprises alight chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NOS: SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5. SEQ ID NO: 13, or SEQ ID NO: 18, and a heavy chain complementarity determining region 3 (CDR3) having an amino acid sequence at least 80% identical to one of SEQ ID NO: 6, or SEQ ID NO: 14. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 1 (CDR1) having an amino acid sequence at least 80% identical to SEQ ID NO: 17, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO: 20. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19; and a heavy chain variable domain (VH) comprising an amino acid sequence at least 80% identical to an amino acid sequence set forth as SEQ ID NO: 8, SEQ ID NO: 16, or SEQ ID NO: 20. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole immunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or fragment thereof is humanized or chimeric. In some instances, the monoclonal antibody or antigen-binding fragment thereof reduces or inhibits shedding of soluble MICA protein, soluble MICB protein, or both, thereby reducing level of soluble MICA protein, soluble MICB protein, or both in the individual. In some instances, the individual has a cancer characterized by elevated levels of soluble MICA protein, soluble MICB protein, or both.

Disclosed herein, in certain embodiments, are monoclonal antibodies or an antigen-binding fragments thereof according to any one of disclosures herein for use in treating cancer in an individual in need thereof; wherein the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof according to any one of disclosures herein for use in preparation of a medicament for treating cancer in an individual in need thereof. More particularly, the antibody or an antigen-binding fragment thereof: a) comprises one or more amino acid modifications to enhance effector function compared to the unmodified monoclonal antibody or antigen-binding fragment thereof, and/or b) lacks fucosylation or comprises reduced fucosylation compared to the amount of fucose normally detected for a similar antibody produced by a mammalian expression system.

Disclosed herein, in certain embodiments, are monoclonal antibodies or an antigen-binding fragments thereof that competitively bind to MICA/B with an antibody comprising a light chain variable domain (VL) having an amino acid sequence at least about 80% identical to an amino acid sequence set forth as SEQ ID NO: 27. Disclosed herein, in certain embodiments, are monoclonal antibodies or an antigen-binding fragments thereof that competitively bind to MICA/B with an antibody comprising a heavy chain variable domain (VH) having an amino acid sequence at least about 80% identical to an amino acid sequence set forth as SEQ ID NO: 28; wherein the antibody or an antigen-binding fragment thereof: a) comprises one or more amino acid modifications to enhance effector function compared to the unmodified monoclonal antibody or antigen-binding fragment thereof; and/or b) lacks fucosylation or comprises reduced fucosylation compared to the amount of fucose normally detected for a similar antibody produced by a mammalian expression system.

In certain embodiments, the monoclonal antibody or an antigen-binding fragment thereof and methods described herein, the monoclonal antibody or an antigen-binding fragment thereof induces one or more of complement dependent cytotoxicity (CDC), Fc receptor binding to activate antibody-dependent cellular cytotoxicity (ADCC), and antibody dependent cellular phagocytosis (ADCP) in a target cell. In certain embodiments, the monoclonal antibody or an antigen-binding fragment thereof induces ADCC.

In certain embodiments of the monoclonal antibody or an antigen-binding fragment thereof described herein or the methods described herein, the monoclonal antibody or an antigen-binding fragment thereof comprises a mutation in the Fc region at amino acid 239. In certain embodiments of the monoclonal antibody or an antigen-binding fragment thereof or the methods described herein the monoclonal antibody or an antigen-binding fragment thereof comprises a mutation in the Fc region at amino acid 332. In certain embodiments of the monoclonal antibody or an antigen-binding fragment thereof described herein or the methods described herein, the monoclonal antibody or an antigen-binding fragment thereof comprises a mutation in the Fc region S239D. In certain embodiments of the monoclonal antibody or an antigen-binding fragment thereof described herein or the method described herein, the monoclonal antibody or an antigen-binding fragment thereof comprises a mutation in the Fc region I332D. In certain embodiments of the monoclonal antibody or an antigen-binding fragment thereof described herein or the methods described herein the monoclonal antibody or an antigen-binding fragment thereof comprises a mutation in the Fc region S239D and I332D. In certain embodiments of the monoclonal antibody or an antigen-binding fragment thereof described herein or the methods described herein, the monoclonal antibody or an antigen-binding fragment thereof contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In certain embodiments of the monoclonal antibody or an antigen-binding fragment thereof described herein or the methods described herein, the monoclonal antibody or an antigen-binding fragment thereof comprises one or more amino acid modifications of Table B and/or Table C.

In certain embodiments of the monoclonal antibody or an antigen-binding fragment thereof described herein or the methods described herein, the monoclonal antibody or an antigen-binding fragment thereof comprises one or more amino acid modifications of Table B and/or Table C.

Disclosed herein, in certain embodiments, the cancer is selected from the group including carcinoma, lymphoma, blastoma, melanoma, and leukemia.

Disclosed herein, in certain embodiments, the cancer is selected from the group consisting of squamous cell carcinoma, small cell lung carcinoma, non-small cell lung carcinoma, gastrointestinal cancer, Hodgkin's and non-Hodgkin's lymphoma, pancreatic cancer, glioblastoma, neuroglioma, cervical cancer, ovarian cancer, liver cancer such as hepatic carcinoma, and hepatocellular carcinoma, bladder cancer, breast cancer, myeloma (such as multiple myeloma), salivary gland carcinoma, kidney cancers such as renal cell carcinoma and Wilms' tumor, basal cell carcinoma, melanoma, prostate cancer, vulva cancer, thyroid cancer, testicular cancer, esophageal cancer, and various types of head and neck cancer.

Disclosed herein, in certain embodiments, the cancer is a primary cancer.

Disclosed herein, in certain embodiments, the cancer is a metastatic cancer.

Disclosed herein, in certain embodiments, the monoclonal antibody or antigen-binding fragment thereof increases surface level of MICA protein, MICB protein, or both.

Disclosed herein, in certain embodiments, the monoclonal antibody or antigen-binding fragment thereof inhibits shedding of MICA protein, MICB protein, or both.

Disclosed herein, in certain embodiments, the monoclonal antibody or antigen-binding fragment thereof increases surface level of soluble MICA protein, soluble MICB protein, or both.

Disclosed herein, in certain embodiments, the monoclonal antibody or antigen-binding fragment thereof inhibits shedding of soluble MICA protein, soluble MICB protein, or both.

Disclosed herein, in certain embodiments, are methods of increasing surface levels of soluble MICA protein, soluble MICB protein, or both via inhibition of shedding in an individual in need thereof, comprising administering to the individual an effective amount of a monoclonal antibody or an antigen-binding fragment thereof comprising a light chain complementarity determining region (CDR) having an amino acid sequence at least 80% identical to at least one of the amino acid sequences set forth as SEQ ID NOS: 1-3, 9-11, and 17, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY.

Disclosed herein, in certain embodiments, are methods of increasing surface levels of soluble MICA protein, soluble MICB protein, or both via inhibition of shedding in an individual in need thereof, comprising administering to the individual an effective amount of a monoclonal antibody or an antigen-binding fragment thereof comprising a heavy chain complementarity determining region (CDR) having an amino acid sequence at least 80% identical to at least one of the amino acid sequences set forth as SEQ ID NOS: 4-6, 12-14, and 18, wherein the monoclonal antibody does not have at least one CDR selected from the list comprising: SASQGISNYLN, TSLLHSG, QQYSKFPRT, GYTFTNYGMN, INTYTGEPTYADDFKG, and NYGNYLFDY. In some instances, the monoclonal antibody or antigen-binding fragment thereof comprises (a) a light chain complementarity determining region (CDR) having an amino acid sequence at least 80% identical to at least one of the amino acid sequences set forth as SEQ ID NOS: 1-3, 9-11, and 17; and (b) a heavy chain complementarity determining region (CDR) having an amino acid sequence at least 80% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 90% identical to at least one of the amino acid sequences set forth as SEQ ID NOS: 1-3, 9-11, and 17; and (b) a heavy chain complementarity determining region (CDR) having an amino acid sequence at least 90% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 95% identical to at least one of the amino acid sequences set forth as SEQ ID NOS: 1-3, 9-11, and 17; and (b) a heavy chain complementarity determining region (CDR) having an amino acid sequence at least 95% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence at least 99% identical to at least one of the amino acid sequences set forth as SEQ ID NOS: 1-3, 9-11, and 17; and (b) a heavy chain complementarity determining region (CDR) having an amino acid sequence at least 99% identical to at least one of the amino acid sequences set forth as SEQ ID NOS: 4-6, 12-14, and 18. In some instances, the monoclonal antibody or antigen-binding fragment thereof comprises (a) a light chain complementarity determining region (CDR) having an amino acid sequence 100% identical to at least one of the amino acid sequences set forth as SEQ ID NOS: 1-3, 9-11, and 17; and (b) a heavy chain complementarity determining region (CDR) having an amino acid sequence 100% identical to at least one of the amino acid sequences set forth as SEQ ID NOS: 4-6, 12-14, and 18. In some instances, the monoclonal antibody or antigen-binding fragment thereof comprises alight chain complementarity determining region 1 (CDR1) having an amino acid sequence at least 80% identical to one of SEQ ID NO: 1, SEQ ID NO: 9, or SEQ ID NO: 17, a light chain complementarity determining region 2 (CDR2) having an amino acid sequence at least 80% identical to one of SEQ ID NOS: SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDRT) having an amino acid sequence at least 80% identical to one of SEQ ID NO: 4, or SEQ ID NO: 12, a heavy chain complementarity determining region 2 (CDR2) having an amino acid sequence at least 80% identical to one of SEQ ID NO: 5, SEQ ID NO: 13, or SEQ ID NO: 18, and a heavy chain complementarity determining region 3 (CDR3) having an amino acid sequence at least 80% identical to one of SEQ ID NO: 6, or SEQ ID NO: 14. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a light chain complementarity determining region 1 (CDR1) having an amino acid sequence at least 80% identical to SEQ ID NO: 17, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 6. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, SEQ ID NO: 19. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO:16, or SEQ ID NO: 20. In some instances, the monoclonal antibody or antigen-binding fragment thereof comprises a light chain variable domain (VL) comprising an amino acid sequence at least 80% identical to an amino acid sequence set forth as SEQ ID NO: 7, SEQ ID NO: 15, or SEQ ID NO: 19; and a heavy chain variable domain (VH) comprising an amino acid sequence at least 80% identical to an amino acid sequence set forth as SEQ ID NO: 8, SEQ ID NO: 16, or SEQ ID NO: 20. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof comprises a light chain variable domain (VL) comprising an amino acid sequence at least 80% identical to SEQ ID NO: 19 and a heavy chain variable domain (VH) comprising an amino acid sequence at least 80% identical to SEQ ID NO: 20. In some instances, the monoclonal antibody or antigen-binding fragment thereof comprises a light chain comprising an amino acid sequence at least 80% identical to an amino acid sequence set forth as SEQ ID NO: 21, SEQ ID NO: 23, or SEQ ID NO: 24. In some instances, the monoclonal antibody or antigen-binding fragment thereof comprises a heavy chain comprising an amino acid sequence at least 80% identical to an amino acid sequence set forth as SEQ ID NO: 22, SEQ ID NO: 25, or SEQ ID NO: 26. In some instances, the monoclonal antibody or antigen-binding fragment thereof specifically binds to a MICA protein, a MICB protein, or both MICA and MICB protein. In some instances, the monoclonal antibody or antigen-binding fragment thereof binds to an alpha-3 domain of a MICA protein, a MICB protein, or both MICA and MICB protein. In some instances, the MICA protein is membrane-bound MICA protein, soluble MICA protein, or both. In some instances, the MICB protein is membrane-bound MICB protein, soluble MICB protein, or both. In some instances, the monoclonal antibody or antigen-binding fragment thereof is selected from a whole inmmunoglobulin, an scFv, a Fab, a F(ab′)₂, or a disulfide linked Fv. In some instances, the monoclonal antibody or antigen-binding fragment thereof is an IgG or IgM. In some instances, the monoclonal antibody or fragment thereof is humanized or chimeric. In some instances, the monoclonal antibody or antigen-binding fragment thereof inhibits shedding of soluble MICA protein, soluble MICB protein, or both, thereby increasing surface level of soluble MICA protein, soluble MICB protein, or both in the individual. In some instances, the individual has a cancer characterized by elevated levels of soluble MICA protein, soluble MICB protein, or both.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A-FIG. 1B exemplify binding of antibodies 3F9.E4 (FIG. 1A) and 16F10.C12 (FIG. 1B) to MICA/B alleles by ELISA.

FIG. 2 exemplifies antibody 3F9.E4 binds to the cell surface MICA, evaluated by flow cytometry staining of TRAMP C2 cells transfected with MICA*08 in comparison to parental TC2 cells.

FIG. 3 exemplifies antibodies 3F9.E4, 16F10.C12 and PDI-1 inhibit MICA shedding from PLC/PRF/5 cells.

FIG. 4A-FIG. 4B exemplifies antibodies 3F9.E4 (FIG. 4A) and 16F10.C12 (FIG. 4B) enhance NK-92 cells mediated cytotoxicity of PLC/PRF/5 cells.

FIG. 5 exemplifies antitumor activity of antibody 3F9 against B16/MICA transfectants relative to isotype control (IC).

FIG. 6A-FIG. 6C exemplify higher percentage of NKG2D positive NK cells (FIG. 6A), CD8⁺ T cells (FIG. 6B) and gamma delta T cells (FIG. 6C) are observed in tumor infiltrates (TILs) in 3F9 antibody treated tumors compared to isotype control.

FIG. 7A-FIG. 7B exemplify treatment with 3F9 antibody in B16/MICA in MICA transgenic animals reduces levels of soluble MICA (FIG. 7A) and surface MICA in tumors (FIG. 7B).

FIG. 8A-FIG. 8C exemplify competition binding assay to illustrate that all antibodies bind to a “structural” epitope and antibody 3F9.E4 competitively binds antibody PDI-1 (FIG. 8B) and antibody 16F10 binds a slightly different epitope than PDI-1 and 3F9, but in close proximity (FIG. 8C).

FIG. 9 is a table showing Biacore binding assay parameters used for determining binding affinity of MICA/B antibodies to the indicated Fcγ Receptors as shown in FIG. 10.

FIG. 10 is a table showing the results of Biacore binding assay of the anti-MICA/B wild type antibody (“CLN619-WT”) anti-MICA/B afucosylated antibody (“GLYMAXX”), anti-MICA/B antibody with two heavy chain S239D and I332E (“S239D and I332E”), or Trastuzumab (positive control) to the indicated Fcγ Receptors. The relative binding affinity is indicating according to the following scale: 10⁻¹ to 10⁻⁹M (++++); 10⁻⁷ (+++); 10⁻⁶ (++); 10⁻⁵ (+); detectable binding (+/−); no detectable binding (−).

FIG. 11 is a table showing the results of Biacore binding assay of the 3F9H1L3L anti-MICA/B wild type antibody (“3F9H1L3L”), 3F9H1L3L anti-MICA/B afucosylated antibody (“3F9H1L3L GLYMAXXX”), 3F9H1L3L anti-MICA/B antibody with two heavy chain S239D and I332E (“3F9HIL3L S239D and I332E”), or Trastuzumab (positive control) to FcRn.

FIG. 12 are graphs depicting results of two replicate MICA/B cell surface stabilization experiments in PLC/PRF/5 cancer cells following incubation with anti-MICA/B wild type antibody (“CLN619 WT”), anti-MICA/B afucosylated antibody (“CLN-619 GLYMAXX”), or anti-MICA/B antibody with heavy chain S239D/I332E mutation stained with Alexafluor anti-MICA/B antibody (AF647 MICA/MICB). Graphs at left show mean fluorescence intensity to determine MICA/B bound to PLC/PRF/5 cells. Graphs at right show controls depicting MICA/B bound to Hela cells highly expressing MICA/B that were incubated with isotype control antibody (AF647 Mo IgG2a Iso CtrL) or unstained Hela cells.

FIG. 13 are graphs depicting luminescence, for ADCC activity using the Promega ADCC Bioassay comprising Jurkat cells stably expressing FcγRIIIa and a luciferase gene regulated by the NFAT response element that were co-cultured with either HCT-116 cells or HCC1534 cells incubated with anti-MICA/B wild type antibody (“CLN619”), anti-MICA/B afucosylated antibody (“GLYMAXX”), or anti-MICA/B antibody with two heavy chain S239D and I332E mutations (DM) for 6 h. Data was plotted as relative light units (RLU) versus Log 10 [antibody] and EC50 was calculated by using non-linear fit log (agonist) vs. response—variable slope (four parameters).

FIG. 14 are graphs depicting percent of live HCT-116 target cells after co-incubation anti-MICA/B wild type antibody (“CLN619”) or anti-MICA/B afucosylated antibody and donor PBMC effector cells (“GLYMAXX”) for 14 h at the indicated concentration.

FIGS. 15A and 15B are graphs depicting time to peri-morbidity endpoint (survival) for Balb/c SCID mice treated with an MICA/B wild type antibody (“CLN619”) or anti-MICA/B afucosylated antibody and donor PBMC effector cells (“GLYMAXX”). FIG. 15A depicts a therapeutic dosing. FIG. 15B depicts a prophylactic dosing.

FIG. 16 is a graph depicting the comparison of an in vivo efficacy study was performed in the HCC1534 lung tumor xenograft model implanted in Balb/c SCID mice treated with an MICA/B wild type antibody (“CLN619”) or anti-MICA/B afucosylated antibody and donor PBMC effector cells (“GLYMAXX”).

FIG. 17 is a graph depicting a representative lot of CLN-619 GlymaxX (afucosylated antibody) that was generated using GlymaxX® Technology. Production of non-fucosylated antibodies by co-expression of heterologous GDP-6-deoxy-D-lyxo-4-hexulose reductase.

Afucosylation was measured using mass spectrometry analysis.

### DETAILED DESCRIPTION

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}.

In other words, “x and/or y” means “one or both of x and y”. As another example, “x, y, and/or z” means any element of the seven-element set {(x), (y), (z), (x, y), (x, z), (y, z) (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

Disclosed herein, in some embodiments, are monoclonal antibodies that bind specifically to MICA/B. In some embodiments, MICA/B antibodies herein bind to MICA/B proteins or fragments thereof and modulate immune response in an individual, thereby treating cancer.

Major histocompatibility complex class I-related chain A and B (MICA/B) are two stress-inducible ligands for natural killer cell (NK) receptor NKG2D and play an important role in mediating the cytotoxicity of NK and T cells. Soluble MICA/B shed by diseased cells (e.g. cancer cells) desensitizes NK and T cells through binding of NKG2D receptor, thereby suppressing the immune response. Accordingly, modulation of MICA/B is useful in modulating an immune response in an individual, for example, in an individual suffering from cancer. Antibodies binding to MICA/B and modulating its activity are desirable for the development of novel therapeutics for treatment of cancer.

### Certain Terminology

As used herein “MICA/B” refers to MICA protein, MICB protein or both MICA and MICB proteins, including their variants, isoforms, and species homologs of human MICA/B.

As used herein “antibody” refers to a glycoprotein which exhibits binding specificity to a specific antigen. An antibody often comprises a variable domain and a constant domain in each of a heavy chain and a light chain. Accordingly, most antibodies have a heavy chain variable domain (VH) and a light chain variable domain (VL) that together form the portion of the antibody that binds to the antigen. Within each variable domain are three complementarity determining regions (CDR) which form loops in the heavy chain variable domain (VH) and light chain variable domain (VL) that contact the surface of the antigen. Antibodies herein also include “antigen binding portion” or fragments of the antibody that are capable of binding to the antigen.

As used herein “chimeric” antibodies are antibodies having a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see e.g., Morrison et al., Proc. Natl. Acad. Sci. USA 81:6851-6855 (1984)). “Humanized antibodies” herein refers chimeric antibodies having human sequences substituted in the antibody sequence.

As used herein, “effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP).

As used herein, “enhanced effector function” refers to one or more modifications to the amino acid sequence of the Fc region of an antibody to increase effector functions of the Fc region including, but not limited to, CDC, ADCC and ADCP as compared to the same antibody without the amino acid modification(s).

The terms “recipient”, “individual”, “subject”, “host”, and “patient”, are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with diseases (e.g. cancer). The term “therapeutic effect” refers to the reduction, elimination, or prevention of the disease, symptoms of the disease, or side effects of the disease in the subject.

A “therapeutically effective amount” in some cases means the amount that, when administered to a subject for treating a disease, is sufficient to effect treatment for that disease.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

### MICA/B

Disclosed herein, in some embodiments, are monoclonal antibodies that bind specifically to MICA/B. Further disclosed herein, in some embodiments, are monoclonal antibodies that competitively bind to MICA/B.

Major Histocompatibility Complex (MHC) class I Chain-related gene A and gene B protein (MICA/B) are glycosylated, polymorphic and membrane-anchored non-classical MHC class I proteins. MICA/B are related to MHC class I and have similar domain structure comprising three extra-cellular Ig-like domains (alpha-1, alpha-2 and alpha-3), a transmembrane domain and a C-terminal cytoplasmic tail. However, MICA/B do not associate with ρ2-microglobulin, lack a CD8 binding site and do not present any antigens. MICA/B are ligands to C-type lectin-like activating receptor Natural Killer Group 2D (NKG2D) on immune effector cells, including NK, NKT and both αβ and γδ CD8⁺ T cells. The interaction of MICA/B and NKG2D plays a role in tumor surveillance, and immune response.

MICA/B proteins are expressed normally at low levels in normal cells, but are induced to higher levels in stressed or transformed cells (e.g. cancer cells). The interaction of NKG2D-bearing immune effector cells with stressed or diseased cells expressing MICA/B ligands on the cell surface creates a cellular immune response against the stressed/diseased cell that culminates in the death of the MICA/B expressing cells. In cancer cells, the truncated MICA/B proteins (proteins that lack the transmembrane domain and cytoplasmic tail but retain the three extracellular domain comprising alpha-1, -2 and -3 domains) are frequently shed into the blood by the action of proteases and results in the down-modulation (receptor internalization) of its intended receptor, NKG2D, on effector immune cells. In some instances, MICA/B glycoproteins are produced intracellularly that are not routinely destined to become cell surface membrane-bound, but instead are incorporated within exosomes and released outside the cell where interaction with NKG2D receptors on immune cells occurs. These truncated or soluble MICA/B ligands shed from the surface of cancer cells function like decoy molecules and lead to down-modulation of the NKG2D receptor on immune effector cells such as NK, NKT and various CD8⁺ T cells. In some instances, the formation of soluble MICA/B leads to the unusual situation where the effectors of the innate defense system, whose natural role is to seek and destroy transformed cells, are shut down by the immunosuppressive actions of these decoy ligand molecules, thereby enabling the cancer cells to hide from the immune system and to grow unchecked.

### Treating Cancer

In some embodiments, anti-MICA/B antibodies disclosed herein bind to MICA/B proteins or fragments thereof and modulate immune response in an individual, thereby treating cancer.

The term “treatment” or “treat” as used herein refers to a clinical intervention that alters the natural progression of a disease in subject being treated. The desired therapeutic effects include, but are not limited to, preventing the occurrence of diseases, alleviating symptoms, reducing any direct or indirect pathological outcomes of diseases, preventing metastasis, delaying disease progression, improving or alleviating conditions, and/or improving prognosis.

The terms “cancer”, “tumor”, “cancerous”, and malignant as used herein refer to or describe a physiological disease in mammals that is typically characterized by unregulated cell growth. Examples of cancers that may be treated by the compositions of the present invention include but are not limited to, carcinomas (e.g., adenocarcinomas), lymphomas, blastomas, melanomas, and leukemias. More specific examples of such cancer include, e.g., squamous cell carcinoma, small cell lung carcinoma, non-small cell lung carcinoma, gastrointestinal cancer, Hodgkin's and non-Hodgkin's lymphoma, pancreatic cancer, glioblastoma, neuroglioma, cervical cancer, ovarian cancer, liver cancer such as hepatic carcinoma, and hepatocellular carcinoma, bladder cancer, breast cancer, myeloma (such as multiple myeloma), salivary gland carcinoma, kidney cancers such as renal cell carcinoma and Wilms' tumor, basal cell carcinoma, melanoma, prostate cancer, vulva cancer, thyroid cancer, testicular cancer, esophageal cancer, and various types of head and neck cancer.

As a result of growth and proliferation of cancerous cells, cancerous masses, which are tumors that invade and destroy adjacent tissues and organs, are formed. Malignant tumors are cancers and while they are often resectable, they may and often do regenerate. Cells of malignant tumors can and do invade and destroy nearby tissues and organs. In addition, cancer cells may leave the site of the malignancy and migrate through the blood or lymphatic system to form new lesions in distal organs. This migration of cancer to distal sites is referred to as metastasis.

Hepatocellular carcinoma (HCC) is a primary malignancy of the liver and occurs predominantly in individuals with underlying chronic liver disease and cirrhosis. Tumors progress with local expansion, intrahepatic spread, and distant metastases. Hepatitis B and Hepatitis C predisposes individuals to the development of chronic liver disease and subsequent development of HCC. Obesity, diabetes, and alcohol abuse are some other causes that predispose individuals to the subsequent development of HCC.

### Anti-MICA/B Antibodies

Provided herein are antibodies that specifically bind to MICA/B3 proteins. In some embodiments, anti-MICA/B antibodies comprise at least one heavy chain and anti-MICA/B antibodies comprise at least one light chain. In some embodiments, anti-MICA/B antibodies comprise at least one heavy chain comprising a heavy chain variable domain (VH) and at least one light chain comprising a light chain variable domain (VL). Each VH and VL comprises three complementarity determining regions (CDR). The amino acid sequences of the VH and VL and the CDRs determine the antigen binding specificity and antigen binding strength of the antibody. The amino acid sequences of the Heavy and Light chains, VH and VL and the CDRs are summarized in Table 1.

TABLE 1
Anti-MICA/B Monoclonal Antibody Sequences

| | | SEQ ID |
|---|---|---|
| | SEQUENCE | NO: |
| 3F9.E4 Light | RSSKSLLHSNGNTYLY | 1 |
| Chain CDR1 | | |
| 3F9.E4 Light | RMSNLAS | 2 |
| Chain CDR2 | | |
| 3F9.E4 Light | MQHLEYPFT | 3 |
| Chain CDR3 | | |
| 3F9.E4 Heavy | GFTFSNYAMS | 4 |
| Chain CDR1 | | |
| 3F9.E4 Heavy | YISPGGDYIYYADTVKG | 5 |
| Chain CDR2 | | |
| 3F9.E4 Heavy | DRRHYGSYAMDY | 6 |
| Chain CDR3 | | |
| 3F9.E4 Light | DIVMTQAAPSVPVTPGESVSISCRSSKSLLHSNGNTYLYWFLQRPGQS | 7 |
| Chain Variable | PQLLIYRMSNLASGVPDRFSGSGSGTAFTLRISRVEAEDVGVYYCMQ | |
| domain | HLEYPFTFGSGTKLEIK | |
| 3F9.E4 Heavy | EVQLQESGEGLVKPGGSLKLSCAASGFTFSNYAMSWVRQTPEKRLE | 8 |
| Chain Variable | WVAYISPGGDYIYYADTVKGRFTISRDNARNTLYLQMSSLKSEDTA | |
| domain | MYYCTTDRRHYGSYAMDYWGQGISVTVSS | |
| 16F10.C12 Light | TASSSVSSNYLH | 9 |
| Chain CDR1 | | |
| 16F10.C12 Light | TTSNLAS | 10 |
| Chain CDR2 | | |
| 16F10.C12 Light | HQFHRSPFT | 11 |
| Chain CDR3 | | |
| 16F10.C12 Heavy | GFSLTAFGVN | 12 |
| Chain CDR1 | | |
| 16F10.C12 Heavy | MIWGDGNTDYNSTLRS | 13 |
| Chain CDR2 | | |
| 16F10.C12 Heavy | ETYYGNYAGLGY | 14 |
| Chain CDR3 | | |
| 16F10.C12 Light | QIVLTQSPAIMSASIGERVTMTCTASSSVSSNYLHWYQQKPRSSPKL | 15 |
| Chain Variable | WIYTTSNLASGVPTRFSGSGSGTSYSLTISSMEAEDAATYYCHQFHRS | |
| domain | PFTFGSGTKLEIK | |
| 16F10.C12 Heavy | EVQLQESGPGLVAPSQSLSITCTVSGFSLTAFGVNWVRQPPGKGLEW | 16 |
| Chain Variable | LGMIWGDGNTDYNSTLRSRLSISKDNSKSQVFLKLNSLQTDDTARYF | |
| domain | CARETYYGNYAGLGYWGQGTLVTVSA | |
| 3F9H1L3L Light | RSSKSLLHSNLNTYLY | 17 |
| Chain CDR1 | | |
| 3F9H1L3L Light | RMSNLAS | 2 |
| Chain CDR2 | | |
| 3F9H1L3L Light | MQHLEYPFT | 3 |
| Chain CDR3 | | |
| 3F9H1L3L Heavy | GFTFSNYAMS | 4 |
| Chain CDR1 | | |
| 3F9H1L3L Heavy | YISPGGDYIYYADSVKG | 18 |
| Chain CDR2 | | |
| 3F9H1L3L Heavy | DRRHYGSYAMDY | 6 |
| Chain CDR3 | | |
| 3F9H1L3L Light | DIVMTQSPLSLPVTPGEPASISCRSSKSLLHSNLNTYLYWFLQKPGQS | 19 |
| Chain Variable | PQILIYRMSNLASGVPDRFSGSGSGTAFTLKISRVEAEDVGVYYCMQ | |
| domain | HLEYPFTFGPGTKLEIKR | |
| 3F9H1L3L Heavy | QVQLVESGGGLVKPGGSLRLSCAASGFTFSNYAMSWIRQAPGKGLE | 20 |
| Chain Variable | WVSYISPGGDYIYYADSVKGRFTISRDNAKNSLYLQMNSLRAEDTA | |
| domain | VYYCTTDRRHYGSYAMDYWGQGTLVTVSS | |
| 3F9H1L3L Light | DIVMTQSPLSLPVTPGEPASISCRSSKSLLHSNLNTYLYWFLQKPGQS | 21 |
| Chain | PQILIYRMSNLASGVPDRFSGSGSGTAFTLKISRVEAEDVGVYYCMQ | |
| | HLEYPFTFGPGTKLEIKRTVAAPSVFIFPPSDEQLKSGTASVVCLLNNF | |
| | YPREAKVQWKVDNALQSGNSQESVTEQDSKDSTYSLSSTLTLSKAD | |
| | YEKHKVYACEVTHQGLSSPVTKSFNRGEC* | |
| 3F9H1L3L Heavy | QVQLVESGGGLVKPGGSLRLSCAASGFTFSNYAMSWIRQAPGKGLE | 22 |
| Chain | WVSYISPGGDYIYYADSVKGRFTISRDNAKNSLYLQMNSLRAEDTA | |
| | VYYCTTDRRHYGSYAMDYWGQGTLVTVSSASTKGPSVFPLAPSSKS | |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPP | |
| | CPAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPG** | |
| 3F9H1L3L Heavy | QVQLVESGGGLVKPGGSLRLSCAASGFTFSNYAMSWIRQAPG | 29 |
| Chain with | KGLEWVSYISPGGDYIYYADSVKGRFTISRDNAKNSLYLQMNS | |
| Exemplary | LRAEDTAVYYCTTDRRHYGSYAMDYWGQGTLVTVSSASTKG | |
| Enhanced Fc | PSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPDVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPEEKTI | |
| | SKAKGQPREPQVYTLPPSREEMTKNQVSLTCLVKGFYPSDIAV | |
| | EWESNGQPENNYKTTPPVLDSDGSFFLYSKLTVDKSRWQQGN | |
| | VFSCSVMHEALHNHYTQKSLSLSPG | |
| PDI-1 humanized | METDTLLLWVLLLWVPGSTGDIQMTQSPSTLSASVGDRVTITCSASQ | 23 |
| LC5 Light Chain | GISNYLNWYQQKPGKAPKLLIQYTSLLHSGVPSRFSGSGSGTEYTLTI | |
| | SSLQPDDFATYFCQQYSKFPRTFGGGTKVEIKRTVAAPSVFIFPPSDE | |
| | QLKSGTASVVCLLNNFYPREAKVQWKVDNALQSGNSQESVTEQDS | |
| | KDSTYSLSSTLTLSKADYEKHKVYACEVTHQGLSSPVTKSFNRGEC* | |
| PDI-1 humanized | METDTLLLWVLLLWVPGSTGDIQMTQSPSSLSASVGDRVTITCSASQ | 24 |
| LC4 Light Chain | GISNYLNWYQQKPGKAPKLLIQYTSLLHSGVPSRFSGSGSGTDYTLTI | |
| | SSLQPEDFATYFCQQYSKFPRTFGGGTKVEIKRTVAAPSVFIFPPSDEQ | |
| | LKSGTASVVCLLNNFYPREAKVQWKVDNALQSGNSQESVTEQDSK | |
| | DSTYSLSSTLTLSKADYEKHKVYACEVTHQGLSSPVTKSFNRGEC* | |
| PDI-1 humanized | MDPKGSLSWRILLFLSLAFELSYGQIQLVQSGSELKKPGASVKVSCK | 25 |
| HC4 Heavy | AFGYTFTNYGMNWVKQAPGQGLKWMGWINTYTGEPTYAQGFTGR | |
| Chain | FVFSLETSVSTAYLQISSLKAEDTAVYFCARNYGNYLFDYWGQGTL | |
| | VTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISR | |
| | TPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREP | |
| | QVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQ | |
| | KSLSLSPG* | |
| PDI-1 humanized | MDPKGSLSWRILLFLSLAFELSYGQIQLVQSGAEVKKPGASVKVSCK | 26 |
| HC6 Heavy | AFGYTFTNYGMNWVKQAPGQGLKWMGWINTYTGEPTYADDFKGR | |
| Chain | VTFTLETSISTAYMELSRLRSDDTAVYFCARNYGNYLFDYWGQGTL | |
| | VTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISR | |
| | TPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREP | |
| | QVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQ | |
| | KSLSLSPG* | |
| PDI-1 Light | DIQMTQTTSSLSASLGDRVTISCSASQGISNYLNWYQQKPDGTLKLLI | 27 |
| Chain Variable | QYTSLLHSGVPSRFSGSGSGTEYSLTISNLEPEDIATYFCQQYSKFPRT | |
| domain | FGGGTKLEIKR | |
| PDI-1 Heavy | QIQLVQSGPELKKSGETVKISCKAFGYTFTNYGMNWVKQAPGKGLK | 28 |
| Chain Variable | WMGWINTYTGEPTYADDFKGRFAFSLETSASTAYLQINHLKNEDTA | |
| domain | TYFCARNYGNYLFDYWGQGTTLTVSS | |

In some embodiments, the antibodies specifically bind to a MICA protein. In some embodiments, the antibodies specifically bind to a MICB protein. In some embodiments, the antibodies specifically bind to both MICA and MICB protein. In some embodiments, the antibodies bind to an alpha-3 domain of a MICA protein. In some embodiments, the antibodies bind to an alpha-3 domain of a MICB protein. In some embodiments, the antibodies bind to an alpha-3 domain of both MICA and MICB protein. In some embodiments, the antibodies bind to a MICA protein that is membrane-bound MICA protein. In some embodiments, the antibodies bind to a MICA protein that is soluble MICA protein. In some embodiments, the antibodies bind to a MICA protein that is both membrane-bound MICA protein and soluble MICA protein. In some embodiments, the antibodies bind to a MICB protein that is membrane-bound MICB protein. In some embodiments, the antibodies bind to a MICB protein that is soluble MICB protein. In some embodiments, the antibodies bind to a MICB protein that is both membrane-bound MICB protein and soluble MICB protein.

In some embodiments, antibodies that specifically bind to MICA/B are monoclonal antibodies. In some embodiments, the antibody is an antigen binding fragment. In some embodiments, the antibody is selected from a whole immunoglobulin, an scFv, a Fab, a F(ab′)2, or a disulfide linked Fv. In some embodiments, the antibody is an IgG or an IgM. In some embodiments, the antibody is humanized. In some embodiments, the antibody is chimeric.

### MICA/B Antibody Heavy and Light Chain

Disclosed herein are antibodies that specifically bind to MICA/B having a light chain. In some embodiments, antibodies binding to MICA/B comprise a light chain having an amino acid sequence at least about 70% identical to an amino acid sequence set forth as SEQ ID NO: 21, SEQ ID NO: 23, or SEQ ID NO: 24. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, SEQ ID NO: 23, or SEQ ID NO: 24. In some embodiments, the light chain has an amino acid sequence 100% identical to an amino acid sequence set forth as SEQ ID NO: 21, SEQ ID NO: 23, or SEQ ID NO: 24.

Further disclosed herein are antibodies that specifically bind to MICA/B having a heavy chain. In some embodiments, antibodies binding to MICA/B comprise a heavy chain having an amino acid sequence at least about 70% identical to an amino acid sequence set forth as SEQ ID NO: 22, SEQ ID NO: 25, or SEQ ID NO: 26. In some embodiments the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, SEQ ID NO: 25, or SEQ ID NO: 26. In some embodiments, the heavy chain has an amino acid sequence 100% identical to an amino acid sequence set forth as SEQ ID NO: 22, SEQ ID NO: 25, or SEQ ID NO: 26.

Also disclosed herein are antibodies binding to MICA/B comprising a light chain and a heavy chain. In some embodiments, antibodies binding to MICA/B comprise a light chain having an amino acid sequence at least about 70% identical to an amino acid sequence set forth as SEQ ID NO: 21, SEQ ID NO: 23, or SEQ ID NO: 24 and a heavy chain having an amino acid sequence at least about 70% identical to an amino acid sequence set forth as SEQ ID NO: 22, SEQ ID NO: 25, or SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, SEQ ID NO: 23, or SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, SEQ ID NO: 25, or SEQ ID NO: 26. In some embodiments, the light chain has an amino acid sequence 100% identical to an amino acid sequence set forth as SEQ ID NO: 21, SEQ ID NO: 23, or SEQ ID NO: 24 and the heavy chain has an amino acid sequence 100% identical to an amino acid sequence set forth as SEQ ID NO: 22, SEQ ID NO: 25, or SEQ ID NO: 26.

In some embodiments, antibodies binding to MICA/B comprise a light chain having an amino acid sequence at least about 70% identical to an amino acid sequence set forth as SEQ ID NO: 21 and a heavy chain having an amino acid sequence at least about 70% identical to an amino acid sequence set forth as SEQ ID NO: 22. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22. In some embodiments, the light chain has an amino acid sequence 100% identical to an amino acid sequence set forth as SEQ ID NO: 21 and the heavy chain has an amino acid sequence 100% identical to an amino acid sequence set forth as SEQ ID NO: 22.

In some embodiments, antibodies binding to MICA/B comprise a light chain having an amino acid sequence at least about 70% identical to an amino acid sequence set forth as SEQ ID NO: 23 and a heavy chain having an amino acid sequence at least about 70% identical to an amino acid sequence set forth as SEQ ID NO: 25. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25. In some embodiments, the light chain has an amino acid sequence 100% identical to an amino acid sequence set forth as SEQ ID NO: 23 and the heavy chain has an amino acid sequence 100% identical to an amino acid sequence set forth as SEQ ID NO: 25.

In some embodiments, antibodies binding to MICA/B comprise a light chain having an amino acid sequence at least about 70% identical to an amino acid sequence set forth as SEQ ID NO: 23 and a heavy chain having an amino acid sequence at least about 70% identical to an amino acid sequence set forth as SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 26. In some embodiments, the light chain has an amino acid sequence 100% identical to an amino acid sequence set forth as SEQ ID NO: 23 and the heavy chain has an amino acid sequence 100% identical to an amino acid sequence set forth as SEQ ID NO: 26.

In some embodiments, antibodies binding to MICA/B comprise a light chain having an amino acid sequence at least about 70% identical to an amino acid sequence set forth as SEQ ID NO: 24 and a heavy chain having an amino acid sequence at least about 70% identical to an amino acid sequence set forth as SEQ ID NO: 25. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25. In some embodiments, the light chain has an amino acid sequence 100% identical to an amino acid sequence set forth as SEQ ID NO: 24 and the heavy chain has an amino acid sequence 100% identical to an amino acid sequence set forth as SEQ ID NO: 25.

In some embodiments, antibodies binding to MICA/B comprise a light chain having an amino acid sequence at least about 70% identical to an amino acid sequence set forth as SEQ ID NO: 24 and a heavy chain having an amino acid sequence at least about 70% identical to an amino acid sequence set forth as SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 26. In some embodiments, the light chain has an amino acid sequence 100% identical to an amino acid sequence set forth as SEQ ID NO: 24 and the heavy chain has an amino acid sequence 100% identical to an amino acid sequence set forth as SEQ ID NO: 26.

### MICA/B Antibody Fc Region

The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirely. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fc gamma receptors. In some aspects, the Fc comprises one or more modifications to promote selective binding of Fc-gamma receptors.

Exemplary mutations that alter the binding of FcRs to the Fe are listed below: S298A/E333A/K334A, S298A/E333A/K334A/K326A (Lu Y, Vemes J M, Chiang N, et al. J Immunol Methods. 2011 Feb. 28; 365(1-2):132-41); F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123); F243L (Stewart R, Thom G, Levens M, et al. Protein Eng Des Sel. 2011 Sep.; 24(9):671-8.), S298A/E333A/K334A (Shields R L, Namenuk A K, Hong K, et al. J Biol Chem. 2001 Mar. 2; 276(9):6591-604): S239D/I332E/A330L, S239D/I332E (Lazar G A, Dang W, Karki S, et al. Proc Natl Acad Sci USA. 2006 Mar. 14; 103(11):4005-10); S239D/S267E, S267E/L328F (Chu S Y, Vostiar I, Karki S, et al. Mol Immunol. 2008 September; 45(15):3926-33), Ser298Ala/Glu333Ala/Lys334Ala, Ser239Asp/Ala330Leu/Ile332Glu, Ser239Asp/Ile332Glu, Gly236Ala/Ser239Asp/Ala330Leu/Ile332Glu, Gly236Ala, Leu234Tyr/Gly236Trp/Ser298Ala, Phe243Leu/Arg292Pro/Tyr300Leu/Va1305Ile/Pro396Leu, Lys326Trp/Glu333Ser, Lys326Ala/Glu333Ala, Lys326Met/Glu333Ser, Cys221Asp/Asp222Cys, Ser267Glu/His268Phe/Ser324Thr, His268Phe/Ser324Thr, Glu345Arg, and other mutations listed in (Saunders K O. Front Immunol. 2019 Jun. 7; 10:1296); S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, Oct. 2012) lists mutations on page 283.

In certain embodiments, the MICA/B antibodies disclosed herein comprise one or more mutations at amino acid positions in the Fc region comprising mutations at amino acid positions 239 and/or 332 of the heavy chain. In certain embodiments, the MICA/B antibodies comprise a S239D and/or a I332E mutation.

In some embodiments, an antibody described herein includes one or more modifications to improve its ability to mediate effector function. Such modifications are known in the art and include afucosylation, or engineering of the affinity of the Fc towards an activating receptor, mainly FCGR3a for ADCC, and towards C1q for CDC. The following Table B summarizes various designs reported in the literature for effector function engineering. Thus, in an embodiment, an antibody described herein can include a dimeric Fc that comprises one or more amino acid modifications as noted in Table B that confer improved effector function.

TABLE B
CH2 domains and effector function engineering

| Reference | Mutations | Effect |
|---|---|---|
| Lu, 2011, | Afucosylated | Increased |
| Ferrara 2011, | | ADCC |
| Mizushima 2011 | | |
| Lu, 2011 | S298A/E333A/K334A | Increased |
| | | ADCC |
| Lu, 2011 | S298A/E333A/K334A/K326A | Increased |
| | | ADCC |
| Stavenhagen, 2007 | F243L/R292P/Y300L/V305I/P396L | Increased |
| | | ADCC |
| Nordstrom, 2011 | F243L/R292P/Y300L/L235V/P396L | Increased |
| | | ADCC |
| Stewart, 2011 | F243L | Increased |
| | | ADCC |
| Shields, 2001 | S298A/E333A/K334A | Increased |
| | | ADCC |
| Lazar, 2006 | S239D/I332E/A330L | Increased |
| | | ADCC |
| Lazar, 2006 | S239D/I332E | Increased |
| | | ADCC |
| Bowles, 2006 | AME-D, not specified mutations | Increased |
| | | ADCC |
| Heider, 2011 | 37.1, mutations not disclosed | Increased |
| | | ADCC |
| Moore, 2010 | S267E/H268F/S324T | Increased |
| | | CDC |
| Ahmed et al., | G236A/S239D/A330L/I332E | Increased |
| 2016, | | ADCC |
| Bournazos, | | |
| et al., 2014 | | |
| Mimoto et | L234Y/G236W/S298A | Increased |
| al., 2013 | | ADCC |
| Richardset | S239D/I332E, G236A | Increased |
| al, 2008 | | ADCC and |
| | | ADCP |

Fc modifications reducing FcgR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcgR or complement binding to the Fc.

Specific, non-limiting examples of known amino acid modifications to reduce FcgR or complement binding to the Fc include those identified in the following Table C:

TABLE C
Modifications to reduce FcgR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E.coli production, non glyco |
| Medimune | L234F/L235E/P331S |
| Trubion | Hinge mutant, possibly C226S/P230S |

Afucosylated MICA/B Antibodies and MICA B Antibodies with Reduced Fucose Content

Antibodies with reduced fucose content have been reported to have an increased affinity for Fc receptors, such as, e.g., FcγRIIIa. Accordingly, in certain embodiments, the anti-MICA/B antibodies or antigen-binding fragments thereof described herein have reduced fucose content or no fucose content. Such antibodies can be produced using techniques known to one skilled in the art. For example, the antibodies can be expressed in cells deficient or lacking the ability of fucosylation. In a specific example, cell lines with a knockout of both alleles of α1,6-fucosyltransferase can be used to produce antibodies with reduced fucose content. The POTELLIGENTR™ system (Lonza) is an example of such a system that can be used to produce antibodies with reduced fucose content. In certain embodiments, antibodies or antigen-binding fragments with reduced fucose content or no fucose content can be produced by, e.g.: (i) culturing cells under conditions which prevent or reduce fucosylation; (ii) posttranslational removal of fucose (e.g., with a fucosidase enzyme); (iii) post-translational addition of the desired carbohydrate, e.g., after recombinant expression of a non-glycosylated glycoprotein; or (iv) purification of the glycoprotein so as to select for antibodies or antigen-binding fragments thereof which are not fucosylated. See, e.g., Longmore G D & Schachter H (1982) Carbohydr Res 100: 365-92 and Imai-Nishiya H et al., (2007) BMC Biotechnol. 7: 84 for methods for producing antibodies or antigen-binding fragments thereof with no fucose content or reduced fucose content.

Methods of producing antibodies with little or no fucose on the Fc glycosylation site (Asn 297 EU numbering) without altering the amino acid sequence are well known in the art. The Glymaxx® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 Dec.; 20 (12):1607-18. Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system. In an embodiment, the antibody can be afucosylated to improve effector function.

Methods of producing antibodies with little or no fucose on the Fc glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 Dec.; 20 (12):1607-18.) Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies

Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

### MICA/B Antibody Variable Domain

Disclosed herein are antibodies that specifically bind to MICA/B having a light chain comprising a light chain variable domain (VL). In some embodiments, antibodies binding to MICA/B comprise a light chain variable domain (VL) having an amino acid sequence at least about 70% identical to an amino acid sequence set forth as SEQ ID NO: 7, SEQ ID NO: 15, or SEQ ID NO: 19. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, SEQ ID NO: 15, or SEQ ID NO: 19. In some embodiments, the VL has an amino acid sequence 100% identical to an amino acid sequence set forth as SEQ ID NO: 7, SEQ ID NO: 15, or SEQ ID NO: 19.

Further disclosed herein are antibodies that specifically bind to MICA/B having a heavy chain comprising a heavy chain variable domain (VH). In some embodiments, antibodies binding to MICA/B comprise a heavy chain variable domain (VH) having an amino acid sequence at least about 70% identical to an amino acid sequence set forth as SEQ ID NO: 8, SEQ ID NO: 16, or SEQ ID NO: 20. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, or SEQ ID NO: 20. In some embodiments, the VH has an amino acid sequence 100% identical to an amino acid sequence set forth as SEQ ID NO: 8, SEQ ID NO: 16, or SEQ ID NO: 20.

Also disclosed herein are antibodies binding to MICA/B comprising a light chain variable domain (VL) and a heavy chain variable domain (VH). In some embodiments, antibodies binding to MICA/B comprise a light chain variable domain (VL) having an amino acid sequence at least about 70% identical to an amino acid sequence set forth as SEQ ID NO: 7, SEQ ID NO: 15, or SEQ ID NO: 19 and a heavy chain variable domain (VH) having an amino acid sequence at least about 70% identical to an amino acid sequence set forth as SEQ ID NO: 8, SEQ ID NO: 16, or SEQ ID NO: 20. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, SEQ ID NO: 15, or SEQ ID NO: 19 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, or SEQ ID NO: 20. In some embodiments, the VL has an amino acid sequence 100% identical to an amino acid sequence set forth as SEQ ID NO: 7, SEQ ID NO: 15, or SEQ ID NO: 19 and the VH has an amino acid sequence 100% identical to an amino acid sequence set forth as SEQ ID NO: 8, SEQ ID NO: 16, or SEQ ID NO: 20.

In some embodiments, antibodies binding to MICA/B comprise a light chain variable domain (VL) having an amino acid sequence at least about 70% identical to an amino acid sequence set forth as SEQ ID NO: 7 and a heavy chain variable domain (VH) having an amino acid sequence at least about 70% identical to an amino acid sequence set forth as SEQ ID NO: 8. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VL has an amino acid sequence 100% identical to an amino acid sequence set forth as SEQ ID NO: 7 and the VH has an amino acid sequence 100% identical to an amino acid sequence set forth as SEQ ID NO: 8.

In some embodiments, antibodies binding to MICA/B comprise a light chain variable domain (VL) having an amino acid sequence at least about 70% identical to an amino acid sequence set forth as SEQ ID NO: 15 and a heavy chain variable domain (VH) having an amino acid sequence at least about 70% identical to an amino acid sequence set forth as SEQ ID NO: 16. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 16. In some embodiments, the VL has an amino acid sequence 100% identical to an amino acid sequence set forth as SEQ ID NO: 15 and the VH has an amino acid sequence 100% identical to an amino acid sequence set forth as SEQ ID NO: 16.

In some embodiments, antibodies binding to MICA/B comprise a light chain variable domain (VL) having an amino acid sequence at least about 70% identical to an amino acid sequence set forth as SEQ ID NO: 19 and a heavy chain variable domain (VH) having an amino acid sequence at least about 70% identical to an amino acid sequence set forth as SEQ ID NO: 20. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 19 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 20. In some embodiments, the VL has an amino acid sequence 100% identical to an amino acid sequence set forth as SEQ ID NO: 19 and the VH has an amino acid sequence 100% identical to an amino acid sequence set forth as SEQ ID NO: 20.

### MICA/B Antibody Complementarity Determining Regions

Disclosed herein are antibodies that specifically bind to MICA/B having a light chain comprising a light chain complementarity determining region (CDR). In some embodiments, antibodies binding to MICA/B comprise a light chain CDR sequence having an amino acid sequence at least about 70% identical to at least one of the amino acid sequences set forth as SEQ ID NOS: 1-3, 9-11, and 17. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, 9-11, and 17. In some embodiments, antibodies binding to MICA/B comprise a light chain CDR sequence having an amino acid sequence 100% identical to at least one of the amino acid sequences set forth as SEQ ID NOS: 1-3, 9-11, and 17.

Further disclosed herein are antibodies that specifically bind to MICA/B having a heavy chain comprising a heavy chain complementarity determining region (CDR). In some embodiments, antibodies binding to MICA/B comprise a heavy chain CDR sequence having an amino acid sequence at least about 70% identical to at least one of the amino acid sequences set forth as SEQ ID NOS: 4-6, 12-14, and 18. In some embodiments, antibodies binding to MICA/B comprise a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, and 18.

In some embodiments, antibodies binding to MICA/B comprise a heavy chain CDR sequence having an amino acid sequence 100% identical to at least one of the amino acid sequences set forth as SEQ ID NOS: 4-6, 12-14, and 18.

Also disclosed herein are antibodies binding to MICA/B comprising a light chain complementarity determining region (CDR) and a heavy chain complementarity determining region (CDR). In some embodiments, antibodies binding to MICA/B comprise a light chain CDR sequence having an amino acid sequence at least about 70% identical to at least one of the amino acid sequences set forth as SEQ ID NOS: 1-3, 9-11, and 17 and a heavy chain CDR sequence having an amino acid sequence at least about 70% identical to at least one of the amino acid sequences set forth as SEQ ID NOS: 4-6, 12-14, and 18. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, 9-11, and 17 and a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, and 18. In some embodiments, antibodies binding to MICA/B comprise a light chain CDR sequence having an amino acid sequence 100% identical to at least one of the amino acid sequences set forth as SEQ ID NOS: 1-3, 9-11, and 17 and a heavy chain CDR sequence having an amino acid sequence at least about 100% identical to at least one of the amino acid sequences set forth as SEQ ID NOS: 4-6, 12-14, and 18.

In some embodiments, antibodies binding to MICA/B comprise a light chain complementarity determining region 1 (CDR1) having an amino acid sequence at least about 70% identical to one of SEQ ID NO: 1, SEQ ID NO: 9, or SEQ ID NO: 17, a light chain complementarity determining region 2 (CDR2) having an amino acid sequence at least about 70% identical to one of SEQ ID NOS: SEQ ID NO: 2, or SEQ ID NO: 10, a light chain complementarity determining region 3 (CDR3) having an amino acid sequence at least about 70% identical to one of SEQ ID NO: 3, or SEQ ID NO: 11, a heavy chain complementarity determining region 1 (CDR1) having an amino acid sequence at least about 70% identical to one of SEQ ID NO: 4, or SEQ ID NO: 12, a heavy chain complementarity determining region 2 (CDR2) having an amino acid sequence at least about 70% identical to one of SEQ ID NO: 5. SEQ ID NO: 13, or SEQ ID NO: 18, and a heavy chain complementarity determining region 3 (CDR3) having an amino acid sequence at least about 70% identical to one of SEQ ID NO: 6, or SEQ ID NO: 14. In some embodiments, antibodies binding to MICA/B comprise a light chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 1, SEQ ID NO: 9, or SEQ ID NO: 17, a light chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NOS: SEQ ID NO: 2, or SEQ ID NO: 10, a light chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 3, or SEQ ID NO: 11, a heavy chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 4, or SEQ ID NO: 12, a heavy chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 5, SEQ ID NO: 13, or SEQ ID NO: 18, and a heavy chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 6, or SEQ ID NO: 14. In some embodiments, antibodies binding to MICA/B comprise a light chain complementarity determining region 1 (CDR1) having an amino acid sequence 100% identical to one of SEQ ID NO: 1, SEQ ID NO: 9, or SEQ ID NO: 17, a light chain complementarity determining region 2 (CDR2) having an amino acid sequence 100% identical to one of SEQ ID NOS: SEQ ID NO: 2, or SEQ ID NO: 10, a light chain complementarity determining region 3 (CDR3) having an amino acid sequence 100% identical to one of SEQ ID NO: 3, or SEQ ID NO: 11, a heavy chain complementarity determining region 1 (CDR1) having an amino acid sequence 100% identical to one of SEQ ID NO: 4, or SEQ ID NO: 12, a heavy chain complementarity determining region 2 (CDR2) having an amino acid sequence 100% identical to one of SEQ ID NO: 5, SEQ ID NO: 13, or SEQ ID NO: 18, and a heavy chain complementarity determining region 3 (CDR3) having an amino acid sequence 100% identical to one of SEQ ID NO: 6, or SEQ ID NO: 14.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies binding to MICA/B comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, and a light chain CDR3 having an amino acid sequence at least about 70% identical to an amino acid sequence set forth as SEQ ID NO: 11. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11. In some embodiments, antibodies binding to MICA/B comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, and a light chain CDR3 having an amino acid sequence 100% identical to an amino acid sequence set forth as SEQ ID NO: 11.

In some embodiments, antibodies binding to MICA/B comprise at least one of a light chain CDR1 having an amino acid sequence at least about 70% identical to an amino acid sequence set forth as SEQ ID NO: 17, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, alight chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies binding to MICA/B comprise at least one of a light chain CDR1 having an amino acid sequence 100% identical to an amino acid sequence set forth as SEQ ID NO: 17, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies binding to MICA/B comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, a heavy chain CDR3 having an amino acid sequence at least about 70% identical to an amino acid sequence set forth as SEQ ID NO: 6. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, a heavy chain CDR3 having an amino acid sequence 100% identical to an amino acid sequence set forth as SEQ ID NO: 6.

In some embodiments, antibodies binding to MICA/B comprise at least one of a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, a heavy chain CDR3 having an amino acid sequence at least about 70% identical to an amino acid sequence set forth as SEQ ID NO: 14. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies binding to MICA/B comprise at least one of a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, a heavy chain CDR3 having an amino acid sequence 100% identical to an amino acid sequence set forth as SEQ ID NO: 14.

In some embodiments, antibodies binding to MICA/B comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 18, a heavy chain CDR3 having an amino acid sequence at least about 70% identical to an amino acid sequence set forth as SEQ ID NO: 6. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 18, a heavy chain CDR3 having an amino acid sequence 100% identical to an amino acid sequence set forth as SEQ ID NO: 6.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, and a heavy chain CDR3 having an amino acid sequence at least about 70% identical to an amino acid sequence set forth as SEQ ID NO: 6. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, and a heavy chain CDR3 having an amino acid sequence 100% identical to an amino acid sequence set forth as SEQ ID NO: 6.

In some embodiments, antibodies binding to MICA/B comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, a light chain CDR3 having an amino acid sequence at least about 70% identical to an amino acid sequence set forth as SEQ ID NO: 11, a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, and a heavy chain CDR3 having an amino acid sequence at least about 70% identical to an amino acid sequence set forth as SEQ ID NO: 14. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies binding to MICA/B comprise at least one of alight chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, a light chain CDR3 having an amino acid sequence 100% identical to an amino acid sequence set forth as SEQ ID NO: 11, a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, and a heavy chain CDR3 having an amino acid sequence 100% identical to an amino acid sequence set forth as SEQ ID NO: 14.

In some embodiments, antibodies binding to MICA/B comprise at least one of a light chain CDR1 having an amino acid sequence at least about 70% identical to an amino acid sequence set forth as SEQ ID NO: 17, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 18, and a heavy chain CDR3 having an amino acid sequence at least about 70% identical to an amino acid sequence set forth as SEQ ID NO: 6. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of alight chain CDR1 having an amino acid sequence 100% identical to an amino acid sequence set forth as SEQ ID NO: 17, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 18, and a heavy chain CDR3 having an amino acid sequence 100% identical to an amino acid sequence set forth as SEQ ID NO: 6.

In some embodiments, antibodies binding to MICA/B do not have at least one CDR selected from the list comprising: SASQGISNYLN, TSLLHSG, QQYSKFPRT, GYTFTNYGMN, INTYTGEPTYADDFKG, and NYGNYLFDY.

### Competitive Binding

Disclosed herein, in some embodiments, are antibodies that competitively bind to MICA/B with an antibody comprising a light chain variable domain (VL) having an amino acid sequence at least about 70% identical to an amino acid sequence set forth as SEQ ID NO: 27. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27. In some embodiments, the VL has an amino acid sequence 100% identical to an amino acid sequence set forth as SEQ ID NO: 27.

Disclosed herein, in some embodiments, are antibodies that competitively bind to MICA/B with an antibody comprising a heavy chain variable domain (VH) having an amino acid sequence at least about 70% identical to an amino acid sequence set forth as SEQ ID NO: 28. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the VH has an amino acid sequence 100% identical to an amino acid sequence set forth as SEQ ID NO: 28.

Disclosed herein, in some embodiments, are antibodies that competitively bind to MICA/B with an antibody comprising a light chain variable domain (VL) having an amino acid sequence at least about 70% identical to an amino acid sequence set forth as SEQ ID NO: 27 and a heavy chain variable domain (VH) having an amino acid sequence at least about 70% identical to an amino acid sequence set forth as SEQ ID NO: 28. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the VL has an amino acid sequence 100% identical to an amino acid sequence set forth as SEQ ID NO: 27 and the VH has an amino acid sequence 100% identical to an amino acid sequence set forth as SEQ ID NO: 28.

### Methods of Treatment and Use

Provided herein are methods of treating cancer in an individual in need thereof comprising administration of an anti-MICA/B antibody disclosed herein.

Further provided herein are methods of reducing or increasing levels of soluble MICA/B proteins in an individual in need thereof comprising administration of an anti-MICA/B antibody disclosed herein.

Also provided herein are methods of alleviating or inhibiting the immunosuppressive environment by preventing, blocking, or enhancing the interaction between soluble MICA/B and NKG2D receptors in an individual in need thereof comprising administration of an anti-MICA/B antibody disclosed herein.

In some embodiments, antibodies binding to MICA/B comprise a light chain CDR sequence having an amino acid sequence at least about 70% identical to at least one of the amino acid sequences set forth as SEQ ID NOS: 1-3, 9-11, and 17. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, 9-11, and 17. In some embodiments, antibodies binding to MICA/B comprise a light chain CDR sequence having an amino acid sequence 100% identical to at least one of the amino acid sequences set forth as SEQ ID NOS: 1-3, 9-11, and 17.

In some embodiments, antibodies binding to MICA/B comprise a heavy chain CDR sequence having an amino acid sequence at least about 70% identical to at least one of the amino acid sequences set forth as SEQ ID NOS: 4-6, 12-14, and 18. In some embodiments, antibodies binding to MICA/B comprise a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, and 18. In some embodiments, antibodies binding to MICA/B comprise a heavy chain CDR sequence having an amino acid sequence 100% identical to at least one of the amino acid sequences set forth as SEQ ID NOS: 4-6, 12-14, and 18.

In some embodiments, antibodies binding to MICA/B comprise a light chain CDR sequence having an amino acid sequence at least about 70% identical to at least one of the amino acid sequences set forth as SEQ ID NOS: 1-3, 9-11, and 17 and a heavy chain CDR sequence having an amino acid sequence at least about 70% identical to at least one of the amino acid sequences set forth as SEQ ID NOS: 4-6, 12-14, and 18. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, 9-11, and 17 and a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, and 18. In some embodiments, antibodies binding to MICA/B comprise a light chain CDR sequence having an amino acid sequence 100% identical to at least one of the amino acid sequences set forth as SEQ ID NOS: 1-3, 9-11, and 17 and a heavy chain CDR sequence having an amino acid sequence at least about 100% identical to at least one of the amino acid sequences set forth as SEQ ID NOS: 4-6, 12-14, and 18.

In some embodiments, antibodies binding to MICA/B comprise a light chain complementarity determining region 1 (CDR1) having an amino acid sequence at least about 70% identical to one of SEQ ID NO: 1, SEQ ID NO: 9, or SEQ ID NO: 17, a light chain complementarity determining region 2 (CDR2) having an amino acid sequence at least about 70% identical to one of SEQ ID NOS: SEQ ID NO: 2, or SEQ ID NO: 10, a light chain complementarity determining region 3 (CDR3) having an amino acid sequence at least about 70% identical to one of SEQ ID NO: 3, or SEQ ID NO: 11, a heavy chain complementarity determining region 1 (CDR1) having an amino acid sequence at least about 70% identical to one of SEQ ID NO: 4, or SEQ ID NO: 12, a heavy chain complementarity determining region 2 (CDR2) having an amino acid sequence at least about 70% identical to one of SEQ ID NO: 5, SEQ ID NO: 13, or SEQ ID NO: 18, and a heavy chain complementarity determining region 3 (CDR3) having an amino acid sequence at least about 70% identical to one of SEQ ID NO: 6, or SEQ ID NO: 14. In some embodiments, antibodies binding to MICA/B comprise alight chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 1, SEQ ID NO: 9, or SEQ ID NO: 17, a light chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NOS: SEQ ID NO: 2, or SEQ ID NO: 10, a light chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 3, or SEQ ID NO: 11, a heavy chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 4, or SEQ ID NO: 12, a heavy chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 5, SEQ ID NO: 13, or SEQ ID NO: 18, and a heavy chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 6, or SEQ ID NO: 14. In some embodiments, antibodies binding to MICA/B comprise a light chain complementarity determining region 1 (CDR1) having an amino acid sequence 100% identical to one of SEQ ID NO: 1, SEQ ID NO: 9, or SEQ ID NO: 17, a light chain complementarity determining region 2 (CDR2) having an amino acid sequence 100% identical to one of SEQ ID NOS: SEQ ID NO: 2, or SEQ ID NO: 10, a light chain complementarity determining region 3 (CDR3) having an amino acid sequence 100% identical to one of SEQ ID NO: 3, or SEQ ID NO: 11, a heavy chain complementarity determining region 1 (CDR1) having an amino acid sequence 100% identical to one of SEQ ID NO: 4, or SEQ ID NO: 12, a heavy chain complementarity determining region 2 (CDR2) having an amino acid sequence 100% identical to one of SEQ ID NO: 5, SEQ ID NO: 13, or SEQ ID NO: 18, and a heavy chain complementarity determining region 3 (CDR3) having an amino acid sequence 100% identical to one of SEQ ID NO: 6, or SEQ ID NO: 14.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies binding to MICA/B comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, and a light chain CDR3 having an amino acid sequence at least about 70% identical to an amino acid sequence set forth as SEQ ID NO: 11. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11. In some embodiments, antibodies binding to MICA/B comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, and a light chain CDR3 having an amino acid sequence 100% identical to an amino acid sequence set forth as SEQ ID NO: 11.

In some embodiments, antibodies binding to MICA/B comprise at least one of a light chain CDR1 having an amino acid sequence at least about 70% identical to an amino acid sequence set forth as SEQ ID NO: 17, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies binding to MICA/B comprise at least one of a light chain CDR1 having an amino acid sequence 100% identical to an amino acid sequence set forth as SEQ ID NO: 17, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies binding to MICA/B comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, a heavy chain CDR3 having an amino acid sequence at least about 70% identical to an amino acid sequence set forth as SEQ ID NO: 6. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, a heavy chain CDR3 having an amino acid sequence 100% identical to an amino acid sequence set forth as SEQ ID NO: 6.

In some embodiments, antibodies binding to MICA/B comprise at least one of a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, a heavy chain CDR3 having an amino acid sequence at least about 70% identical to an amino acid sequence set forth as SEQ ID NO: 14. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies binding to MICA/B comprise at least one of a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, a heavy chain CDR3 having an amino acid sequence 100% identical to an amino acid sequence set forth as SEQ ID NO: 14.

In some embodiments, antibodies binding to MICA/B comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 18, a heavy chain CDR3 having an amino acid sequence at least about 70% identical to an amino acid sequence set forth as SEQ ID NO: 6. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 18, a heavy chain CDR3 having an amino acid sequence 100% identical to an amino acid sequence set forth as SEQ ID NO: 6.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, and a heavy chain CDR3 having an amino acid sequence at least about 70% identical to an amino acid sequence set forth as SEQ ID NO: 6. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, and a heavy chain CDR3 having an amino acid sequence 100% identical to an amino acid sequence set forth as SEQ ID NO: 6.

In some embodiments, antibodies binding to MICA/B comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, a light chain CDR3 having an amino acid sequence at least about 70% identical to an amino acid sequence set forth as SEQ ID NO: 11, a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, and a heavy chain CDR3 having an amino acid sequence at least about 70% identical to an amino acid sequence set forth as SEQ ID NO: 14. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies binding to MICA/B comprise at least one of alight chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, a light chain CDR3 having an amino acid sequence 100% identical to an amino acid sequence set forth as SEQ ID NO: 11, a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, and a heavy chain CDR3 having an amino acid sequence 100% identical to an amino acid sequence set forth as SEQ ID NO: 14.

In some embodiments, antibodies binding to MICA/B comprise at least one of a light chain CDR1 having an amino acid sequence at least about 70% identical to an amino acid sequence set forth as SEQ ID NO: 17, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 18, and a heavy chain CDR3 having an amino acid sequence at least about 70% identical to an amino acid sequence set forth as SEQ ID NO: 6. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a light chain CDR1 having an amino acid sequence 100% identical to an amino acid sequence set forth as SEQ ID NO: 17, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 18, and a heavy chain CDR3 having an amino acid sequence 100% identical to an amino acid sequence set forth as SEQ ID NO: 6.

In some embodiments, antibodies binding to MICA/B do not have at least one CDR selected from the list comprising: SASQGISNYLN, TSLLHSG, QQYSKFPRT, GYTFTNYGMN, INTYTGEPTYADDFKG, and NYGNYLFDY.

In some embodiments, antibodies binding to MICA/B comprise a light chain variable domain (VL) having an amino acid sequence at least about 70% identical to an amino acid sequence set forth as SEQ ID NO: 7, SEQ ID NO: 15, or SEQ ID NO: 19. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, SEQ ID NO: 15, or SEQ ID NO: 19. In some embodiments, the VL has an amino acid sequence 100% identical to an amino acid sequence set forth as SEQ ID NO: 7, SEQ ID NO: 15, or SEQ ID NO: 19.

In some embodiments, antibodies binding to MICA/B comprise a heavy chain variable domain (VH) having an amino acid sequence at least about 70% identical to an amino acid sequence set forth as SEQ ID NO: 8, SEQ ID NO: 16, or SEQ ID NO: 20. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, or SEQ ID NO: 20. In some embodiments, the VH has an amino acid sequence 100% identical to an amino acid sequence set forth as SEQ ID NO: 8, SEQ ID NO: 16, or SEQ ID NO: 20.

In some embodiments, antibodies binding to MICA/B comprise a light chain variable domain (VL) having an amino acid sequence at least about 70% identical to an amino acid sequence set forth as SEQ ID NO: 7, SEQ ID NO: 15, or SEQ ID NO: 19 and a heavy chain variable domain (VH) having an amino acid sequence at least about 70% identical to an amino acid sequence set forth as SEQ ID NO: 8, SEQ ID NO: 16, or SEQ ID NO: 20. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, SEQ ID NO: 15, or SEQ ID NO: 19 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, or SEQ ID NO: 20. In some embodiments, the VL has an amino acid sequence 100% identical to an amino acid sequence set forth as SEQ ID NO: 7, SEQ ID NO: 15, or SEQ ID NO: 19 and the VH has an amino acid sequence 100% identical to an amino acid sequence set forth as SEQ ID NO: 8, SEQ ID NO: 16, or SEQ ID NO: 20.

In some embodiments, antibodies binding to MICA/B comprise a light chain variable domain (VL) having an amino acid sequence at least about 70% identical to an amino acid sequence set forth as SEQ ID NO: 7 and a heavy chain variable domain (VH) having an amino acid sequence at least about 70% identical to an amino acid sequence set forth as SEQ ID NO: 8. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VL has an amino acid sequence 100% identical to an amino acid sequence set forth as SEQ ID NO: 7 and the VH has an amino acid sequence 100% identical to an amino acid sequence set forth as SEQ ID NO: 8.

In some embodiments, antibodies binding to MICA/B comprise a light chain variable domain (VL) having an amino acid sequence at least about 70% identical to an amino acid sequence set forth as SEQ ID NO: 15 and a heavy chain variable domain (VH) having an amino acid sequence at least about 70% identical to an amino acid sequence set forth as SEQ ID NO: 16. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 16. In some embodiments, the VL has an amino acid sequence 100% identical to an amino acid sequence set forth as SEQ ID NO: 15 and the VH has an amino acid sequence 100% identical to an amino acid sequence set forth as SEQ ID NO: 16.

In some embodiments, antibodies binding to MICA/B comprise a light chain variable domain (VL) having an amino acid sequence at least about 70% identical to an amino acid sequence set forth as SEQ ID NO: 19 and a heavy chain variable domain (VH) having an amino acid sequence at least about 70% identical to an amino acid sequence set forth as SEQ ID NO: 20. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 19 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 20. In some embodiments, the VL has an amino acid sequence 100% identical to an amino acid sequence set forth as SEQ ID NO: 19 and the VH has an amino acid sequence 100% identical to an amino acid sequence set forth as SEQ ID NO: 20.

In some embodiments, antibodies binding to MICA/B comprise a light chain having an amino acid sequence at least about 70% identical to an amino acid sequence set forth as SEQ ID NO: 21, SEQ ID NO: 23, or SEQ ID NO: 24. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, SEQ ID NO: 23, or SEQ ID NO: 24. In some embodiments, the light chain has an amino acid sequence 100% identical to an amino acid sequence set forth as SEQ ID NO: 21, SEQ ID NO: 23, or SEQ ID NO: 24.

In some embodiments, antibodies binding to MICA/B comprise a heavy chain having an amino acid sequence at least about 70% identical to an amino acid sequence set forth as SEQ ID NO: 22, SEQ ID NO: 25, or SEQ ID NO: 26. In some embodiments the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, SEQ ID NO: 25, or SEQ ID NO: 26. In some embodiments, the heavy chain has an amino acid sequence 100% identical to an amino acid sequence set forth as SEQ ID NO: 22, SEQ ID NO: 25, or SEQ ID NO: 26.

Also disclosed herein are antibodies binding to MICA/B comprising a light chain and a heavy chain. In some embodiments, antibodies binding to MICA/B comprise a light chain having an amino acid sequence at least about 70% identical to an amino acid sequence set forth as SEQ ID NO: 21, SEQ ID NO: 23, or SEQ ID NO: 24 and a heavy chain having an amino acid sequence at least about 70% identical to an amino acid sequence set forth as SEQ ID NO: 22, SEQ ID NO: 25, or SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, SEQ ID NO: 23, or SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, SEQ ID NO: 25, or SEQ ID NO: 26. In some embodiments, the light chain has an amino acid sequence 100% identical to an amino acid sequence set forth as SEQ ID NO: 21, SEQ ID NO: 23, or SEQ ID NO: 24 and the heavy chain has an amino acid sequence 100% identical to an amino acid sequence set forth as SEQ ID NO: 22, SEQ ID NO: 25, or SEQ ID NO: 26.

In some embodiments, antibodies binding to MICA/B comprise a light chain having an amino acid sequence at least about 70% identical to an amino acid sequence set forth as SEQ ID NO: 21 and a heavy chain having an amino acid sequence at least about 70% identical to an amino acid sequence set forth as SEQ ID NO: 22. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22. In some embodiments, the light chain has an amino acid sequence 100% identical to an amino acid sequence set forth as SEQ ID NO: 21 and the heavy chain has an amino acid sequence 100% identical to an amino acid sequence set forth as SEQ ID NO: 22.

In some embodiments, antibodies binding to MICA/B comprise a light chain having an amino acid sequence at least about 70% identical to an amino acid sequence set forth as SEQ ID NO: 23 and a heavy chain having an amino acid sequence at least about 70% identical to an amino acid sequence set forth as SEQ ID NO: 25. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25. In some embodiments, the light chain has an amino acid sequence 100% identical to an amino acid sequence set forth as SEQ ID NO: 23 and the heavy chain has an amino acid sequence 100% identical to an amino acid sequence set forth as SEQ ID NO: 25.

In some embodiments, antibodies binding to MICA/B comprise a light chain having an amino acid sequence at least about 70% identical to an amino acid sequence set forth as SEQ ID NO: 23 and a heavy chain having an amino acid sequence at least about 70% identical to an amino acid sequence set forth as SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 26. In some embodiments, the light chain has an amino acid sequence 100% identical to an amino acid sequence set forth as SEQ ID NO: 23 and the heavy chain has an amino acid sequence 100% identical to an amino acid sequence set forth as SEQ ID NO: 26.

In some embodiments, antibodies binding to MICA/B comprise a light chain having an amino acid sequence at least about 70% identical to an amino acid sequence set forth as SEQ ID NO: 24 and a heavy chain having an amino acid sequence at least about 70% identical to an amino acid sequence set forth as SEQ ID NO: 25. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25. In some embodiments, the light chain has an amino acid sequence 100% identical to an amino acid sequence set forth as SEQ ID NO: 24 and the heavy chain has an amino acid sequence 100% identical to an amino acid sequence set forth as SEQ ID NO: 25.

In some embodiments, antibodies binding to MICA/B comprise a light chain having an amino acid sequence at least about 70% identical to an amino acid sequence set forth as SEQ ID NO: 24 and a heavy chain having an amino acid sequence at least about 70% identical to an amino acid sequence set forth as SEQ ID NO: 26. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 24 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 26. In some embodiments, the light chain has an amino acid sequence 100% identical to an amino acid sequence set forth as SEQ ID NO: 24 and the heavy chain has an amino acid sequence 100% identical to an amino acid sequence set forth as SEQ ID NO: 26.

In some embodiments, antibodies competitively bind to MICA/B with an antibody comprising a light chain variable domain (VL) having an amino acid sequence at least about 70% identical to an amino acid sequence set forth as SEQ ID NO: 27. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27. In some embodiments, the VL has an amino acid sequence 100% identical to an amino acid sequence set forth as SEQ ID NO: 27.

In some embodiments, antibodies competitively bind to MICA/B with an antibody comprising a heavy chain variable domain (VH) having an amino acid sequence at least about 70% identical to an amino acid sequence set forth as SEQ ID NO: 28. In some embodiments the VH has an amino acid sequence at least about 75%, 80%, 81%, 82%, 83%, 84%, 85%, 86%, 87%, 88% 89%, 90%, 91%, 92%, 93%, 94%, 95%, 96%, 97%, 98%, or 99% identical to an amino acid sequence set forth as SEQ ID NO: 28. In some embodiments, the VH has an amino acid sequence 100% identical to an amino acid sequence set forth as SEQ ID NO: 28.

In some embodiments, antibodies competitively bind to MICA/B with an antibody comprising a light chain variable domain (VL) having an amino acid sequence at least about 70% identical to an amino acid sequence set forth as SEQ ID NO: 27 and a heavy chain variable domain (VH) having an amino acid sequence at least about 70% identical to an amino acid sequence set forth as SEQ ID NO: 28. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the VL has an amino acid sequence 100% identical to an amino acid sequence set forth as SEQ ID NO: 27 and the VH has an amino acid sequence 100% identical to an amino acid sequence set forth as SEQ ID NO: 28.

In some embodiments, the antibodies specifically bind to a MICA protein. In some embodiments, the antibodies specifically bind to a MICB protein. In some embodiments, the antibodies specifically bind to both MICA and MICB protein. In some embodiments, the antibodies bind to an alpha-3 domain of a MICA protein. In some embodiments, the antibodies bind to an alpha-3 domain of a MICB protein. In some embodiments, the antibodies bind to an alpha-3 domain of both MICA and MICB protein. In some embodiments, the antibodies bind to a MICA protein that is membrane-bound MICA protein. In some embodiments, the antibodies bind to a MICA protein that is soluble MICA protein. In some embodiments, the antibodies bind to a MICA protein that is both membrane-bound MICA protein and soluble MICA protein. In some embodiments, the antibodies bind to a MICB protein that is membrane-bound MICB protein. In some embodiments, the antibodies bind to a MICB protein that is soluble MICB protein. In some embodiments, the antibodies bind to a MICB protein that is both membrane-bound MICB protein and soluble MICB protein.

In some embodiments, antibodies that specifically bind to MICA/B are monoclonal antibodies. In some embodiments, the antibody is an antigen binding fragment. In some embodiments, the antibody is selected from a whole immunoglobulin, an scFv, a Fab, a F(ab′)2, or a disulfide linked Fv. In some embodiments, the antibody is an IgG or an IgM. In some embodiments, the antibody is humanized. In some embodiments, the antibody is chimeric.

In some embodiments, the antibodies disclosed herein reduce level of soluble MICA protein. In some embodiments, the antibodies disclosed herein reduce level of soluble MICB protein.

In some embodiments, the antibodies disclosed herein reduce level of both soluble MICA protein and soluble MICB protein. In some embodiments, the antibodies disclosed herein reduce shedding of soluble MICA protein. In some embodiments, the antibodies disclosed herein reduce shedding of soluble MICB protein. In some embodiments, the antibodies disclosed herein reduce shedding of both soluble MICA protein and soluble MICB protein. In some embodiments, the antibodies disclosed herein inhibit shedding of soluble MICA protein. In some embodiments, the antibodies disclosed herein inhibit shedding of soluble MICB protein. In some embodiments, the antibodies disclosed herein inhibit shedding of both soluble MICA protein and soluble MICB protein.

In some embodiments, the antibodies disclosed herein increase surface level of MICA protein. In some embodiments, the antibodies disclosed herein increase surface level of MICB protein. In some embodiments, the antibodies disclosed herein increase level of both MICA protein and MICB protein. In some embodiments, the antibodies disclosed herein reduce shedding of MICA protein. In some embodiments, the antibodies disclosed herein reduce shedding of MICB protein. In some embodiments, the antibodies disclosed herein reduce shedding of both MICA protein and MICB protein. In some embodiments, the antibodies disclosed herein inhibit shedding of soluble MICA protein. In some embodiments, the antibodies disclosed herein inhibit shedding of soluble MICB protein. In some embodiments, the antibodies disclosed herein inhibit shedding of both soluble MICA protein and soluble MICB protein.

Any suitable route of administration is contemplated for use with the methods disclosed herein. In some embodiments, the antibody is administered by intravenous administration.

In some embodiments, the antibody is administered by subcutaneous administration. In some embodiments, the antibody is administered locally. In some embodiments, the antibody is administered systemically (e.g., intravenously, intramuscularly, subcutaneously, intradermally, orally, intranasally, sublingually). In some embodiments, the antibody is formulated as a salve, lotion or emulsion. In some embodiments, the antibody is formulated as a solution. In some embodiments, the antibody is formulated for topical, oral, buccal, or nasal administration.

In some embodiments, the individual is monitored prior to administration of the antibody. Symptoms are identified and their severity is assessed. An antibody as described herein is administered alone or in combination with additional treatments, singly or multiply over time as discussed herein or known to one of skill in the art. In some embodiments, the individual is monitored such that the efficacy of the treatment regimen is determined. In some embodiments, a treatment regimen is modified in response to preliminary treatment outcomes, such that treatment dose or frequency or dose and frequency is altered so as to attain a desired level of subject response in light of symptom alleviation, side effect reduction, or a combination of symptom alleviation and side effect reduction.

Therapeutically effective amounts or dosages are contemplated to include dosages of about 0.01 mg/kg to about 20 mg/kg, about for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6 mg/kg, about 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8 mg/kg, about 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11 mg/kg, about 11.1 mg/kg, about 11.2 mg/kg, about 11.3 mg/kg, about 11.4 mg/kg, about 11.5 mg/kg, about 11.6 mg/kg, about 11.7 mg/kg, about 11.8 mg/kg, about 11.9 mg/kg, about 12 mg/kg, about 12.1 mg/kg, about 12.2 mg/kg, about 12.3 mg/kg, about 12.4 mg/kg, about 12.5 mg/kg, about 12.6 mg/kg, about 12.7 mg/kg, about 12.8 mg/kg, about 12.9 mg/kg, about 13 mg/kg, about 13.1 mg/kg, about 13.2 mg/kg, about 13.3 mg/kg, about 13.4 mg/kg, about 13.5 mg/kg, about 13.6 mg/kg, about 13.7 mg/kg, about 13.8 mg/kg, about 13.9 mg/kg, about 14 mg/kg, about 14.1 mg/kg, about 14.2 mg/kg, about 14.3 mg/kg, about 14.4 mg/kg, about 14.5 mg/kg, about 14.6 mg/kg, about 14.7 mg/kg, about 14.8 mg/kg, about 14.9 mg/kg, about 15 mg/kg, about 15.1 mg/kg, about 15.2 mg/kg, about 15.3 mg/kg, about 15.4 mg/kg, about 15.5 mg/kg, about 15.6 mg/kg, about 15.7 mg/kg, about 15.8 mg/kg, about 15.9 mg/kg, about 16 mg/kg, about 16.1 mg/kg, about 16.2 mg/kg, about 16.3 mg/kg, about 16.4 mg/kg, about 16.5 mg/kg, about 16.6 mg/kg, about 16.7 mg/kg, about 16.8 mg/kg, about 16.9 mg/kg, about 17 mg/kg, about 17.1 mg/kg, about 17.2 mg/kg, about 17.3 mg/kg, about 17.4 mg/kg, about 17.5 mg/kg, about 17.6 mg/kg, about 17.7 mg/kg, about 17.8 mg/kg, about 17.9 mg/kg, about 18 mg/kg, about 18.1 mg/kg, about 18.2 mg/kg, about 18.3 mg/kg, about 18.4 mg/kg, about 18.5 mg/kg, about 18.6 mg/kg, about 18.7 mg/kg, about 18.8 mg/kg, about 18.9 mg/kg, about 19 mg/kg, about 19.1 mg/kg, about 19.2 mg/kg, about 19.3 mg/kg, about 19.4 mg/kg, about 19.5 mg/kg, about 19.6 mg/kg, about 19.7 mg/kg, about 19.8 mg/kg, about 19.9 mg/kg, about or 20 mg/kg. Therapeutically effective amounts or dosages, in some cases, are contemplated to include dosages of about 0.1 mg/kg to about 2.0 mg/kg.

Methods of treatment herein comprise one or more administrations of anti-MICA/B antibodies in doses disclosed herein. In some embodiments, methods comprise one administration of anti-MICA/B antibodies. In some embodiments, methods comprise two administrations of anti-MICA/B antibodies. In some embodiments, methods comprise three administrations of anti-MICA/B antibodies. In some embodiments, methods comprise four administrations of anti-MICA/B antibodies. In some embodiments, methods comprise five administrations of anti-MICA/B antibodies. In some embodiments, methods comprise six administrations of anti-MICA/B antibodies.

In some embodiments, one or more administrations of anti-MICA/B antibodies are administered daily. In some embodiments, one or more administrations of anti-MICA/B antibodies are administered weekly. In some embodiments, one or more administrations of anti-MICA/B antibodies are administered biweekly. In some embodiments, one or more administrations of anti-MICA/B antibodies are administered every three weeks. In some embodiments, one or more administrations of anti-MICA/B antibodies are administered monthly. In some embodiments, one or more administrations of anti-MICA/B antibodies are administered every three months. In some embodiments, one or more administrations of anti-MICA/B antibodies are administered every six months. In some embodiments, one or more administrations of anti-MICA/B antibodies are administered yearly.

In some embodiments, the methods of treatment disclosed herein, is a monotherapy. In some embodiments, the methods of treatment disclosed herein, is a combination therapy. In some embodiments, combination therapy comprises administrations of anti-MICA/B antibodies in combination with another therapeutic agent. In some embodiments, the therapeutic agent comprises a chemotherapeutic agent. In some embodiments, the chemotherapeutic agent include, but is not limited to, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, kinase inhibitors and immune checkpoint inhibitors. In some embodiments, the anti-MICA/B antibodies disclosed herein are administered in combination with a therapeutic agent that induces an immune response. In some embodiments, the anti-MICA/B antibodies disclosed herein are administered in combination with a therapeutic agent that inhibits downregulation of an immune response. In some embodiments, inducing an immune response comprises activation or upregulating activity of NK cells. In some embodiments, inducing an immune response comprises activation or upregulating activity of T cells. In some embodiments, the immune check point inhibitor target comprises PD-1. In some embodiments, the immune check point inhibitor target comprises PD-LI. In some embodiments, the immune checkpoint inhibitor comprises Pembrolizumab, Nivolumab, Cemiplimab, AMP-224, AMP-514, PDR001, Atezolizumab, Avelumab, Durvalumab, BMS-936559, and CK-301. In certain embodiments, the anti-MICA/B antibodies described herein are administered in combination with radiation therapy. In certain embodiments, the anti-MICA/B antibodies described herein are administered in combination with one or more cell therapies.

### Pharmaceutical Compositions

Also disclosed herein are pharmaceutical compositions comprising anti-MICA/B antibodies disclosed herein and a pharmaceutically acceptable carrier or excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments, the compositions further comprise an additional therapeutic agent. In some embodiments, the therapeutic agent is a chemotherapeutic agent. The chemotherapeutic agents can include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments, the antibody and the therapeutic agent are in the same formulation. In some embodiments, the antibody and the therapeutic agent are in different formulation. In some embodiments, antibody described herein is used prior to the administration of the other therapeutic agent. In some embodiments, antibody described herein is used concurrently with the administration of the other therapeutic agent. In some embodiments, antibody described herein is used subsequent to the administration of the other therapeutic agent.

Pharmaceutical formulations, in some embodiments, are made to be compatible with a particular local, regional or systemic administration or delivery route. Thus, pharmaceutical formulations include carriers, diluents, or excipients suitable for administration by particular routes.

Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Antibody Binding Kinetics Measurement

Affinity kinetics was determined on a ForteBio Octet Red96 analyzer. Briefly, anti-MICA/B mAb (30 μg/ml) was captured on Dip and Read™ Anti-mouse IgG Fc Capture (AMC) Biosensors (ForteBio) at room temperature in an assay buffer of PBS+0.1% BSA+0.02% Tween-20 (pH 7.2). Sensors were washed in assay buffer and then incubated with purified 6×His-MICA*08 or 6×His-MICA*04 proteins (100 nM), in 2-fold dilution series for 5 minutes in assay buffer to determine association kinetics of the antibody with the protein antigen. Sensors were then incubated in assay buffer for 10 minutes to determine dissociation kinetics. The resulting kinetics parameters were calculated with ForteBio analysis suite 8.0 using a 1:1 model. Results for these assays are shown in Table 2.

TABLE 2
Kinetic measurements of anti-MICA/B antibodies
to MICA antigen by BioLayer Interferometry.

| Antibody | KD (M) | kon(1/Ms) | kdis(1/s) |
|---|---|---|---|
| 3F9.E4 | 1.18E−09 | 3.83E+05 | 4.21E−04 |
| 16F10.C12 | 6.45E−09 | 9.33E+04 | 5.48E−04 |

### Example 2. Antibody Binding to MICA/B Alleles

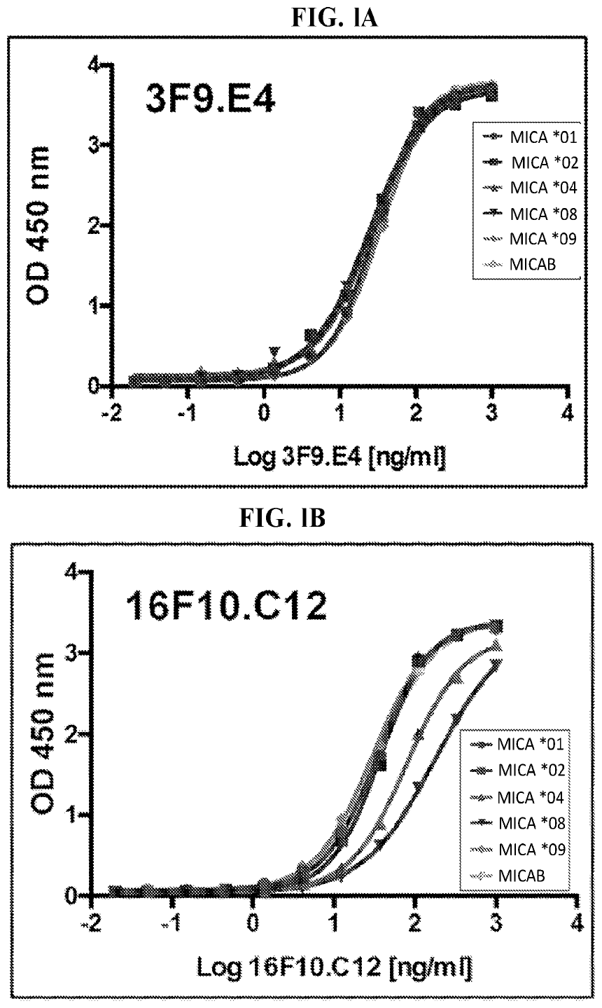

Recombinant MICA*01, MICA*02, MICA*04, MICA*08, MICA*09, and MICB proteins were diluted to 1 μg/ml in 50 mM sodium carbonate buffer, pH 9.6, and coated onto high binding 96-well microplates (Corning #9018), 100 ng in 100 μl per well. The following morning, coated ELISA plates were washed three times with TBS-Tween-20, pH 7.4 and then blocked in SuperBlock T20 Blocking Buffer (Pierce #37536). After blocking, ELISA plates were washed once with TBS-T and incubated with serially diluted anti-MICA antibody (0-1 g/ml) for approximately two hours at room temperature. Following the incubation, ELISA plates were washed three times with TBS-T and then incubated with Goat anti-Mouse IgG (H+L)-HRP conjugate (ThermoFisher Scientific #626520) for 45 minutes at room temperature with shaking (˜400 rpm). After the incubation, ELISA plates were washed three times with TBS-T and incubated with Super Sensitive Liquid Substrate TMB (Sigma #T4444), 100 μl per well, until color development was sufficient. The reaction was stopped with 1N sulfuric acid, 100 μl per well. Optical density (OD) values were measured at 450 nm using a microplate reader. The results of this assay are shown in FIG. 1A-FIG. 1B.

### Example 3. Antibody Binding to Cell Surface MICA

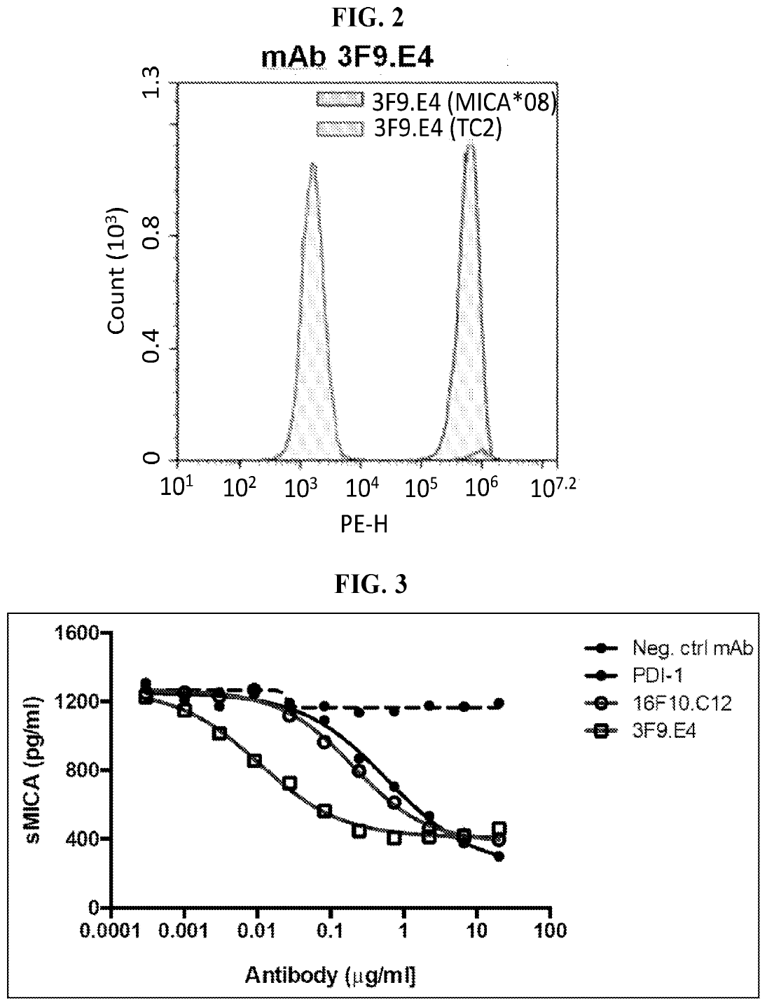

Mouse prostate adenocarcinoma TRAMP-C2 cells (TC2) (ATCC, Manassas, VA) were used to generate a stable cell line expressing MICA*08 allele (TC2-MICA-08). Binding of anti-MICA antibody to TC2-MICA-08 was analyzed by flow cytometry. Briefly, cells were first stained with LIVE/DEAD Near IR Stain (Thermo) for 30 min at 4° C., and then washed once by centrifugation with FACS Buffer (1 mM EDTA, 25 mM HEPES, 2% FBS in 1×PBS). About 2-3×10⁵ TC2-MICA-08 cells were incubated with 100 μl of FACS Buffer containing 500 ng anti-MICA antibody at 4° C. for 30 min, followed by incubation with 100 μl 2 g/ml PE conjugated goat-anti-mouse IgG (Biolegend, San Diego, CA) secondary Ab at 4° C. for 30 min. Cells were then washed once and cell pellet resuspended with FACS Buffer for FACS analysis gated on live cells. Significantly higher PE fluorescence signal was observed with TC2-MICA-08 cells compared to parental TC2 cells indicated binding of anti-MICA mAb to surface expressed MICA. Result for this assay is shown in FIG. 2.

### Example 4. Antibody Inhibits MICA Shedding from PLC/PRF/5 Cells

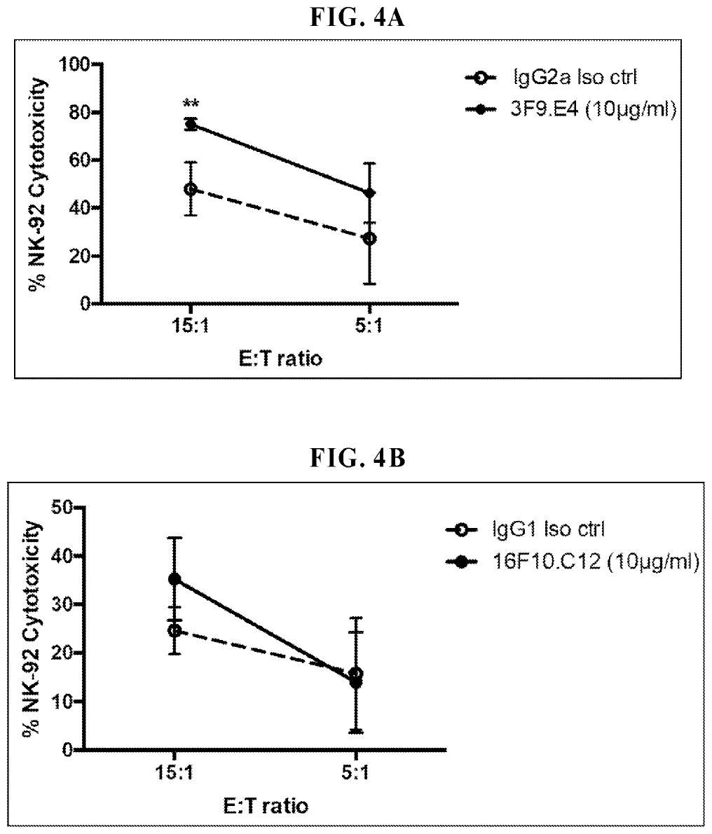

4×10⁴ PLC/PRF/5 cells (Hepatocellular Carcinoma) (ATCC, Manassas, VA) were plated in 96-well plate and incubated at 37° C. overnight. Cells were then treated with 100 μl Complete Media (MEM+10% FBS, Thermo, Grand Island, NY) containing anti-MICA antibody that binds to MICA α3 domain and negative control antibodies, respectively, and incubated at 37° C. for another day. Cell supernatants containing shed MICA were used to determine the level of soluble MICA by ELISA. Briefly, 96-well plate was coated with 100 μl 2 μg/ml anti-human MICA/B (clone BAMO1, MBL, Japan) overnight at 4° C. Plate was blocked and then incubated with cell supernatants and MICA standards for 2 hours. After incubation, plates were washed and followed by 1 hour incubation with 1 μg/ml proprietary anti-human MICA/MICB mAb, clone 10E9 conjugated with biotin. Next, 100 μl HRP conjugated streptavidin (HRP-SA) (R&D Systems, Minneapolis, MN) was added to wells and incubated for 30 min. Samples were developed with TMB for 4 min, stopped with 1N sulfuric acid and detected with absorbance at 450 nm. Soluble MICA level was interpolated from standard curve. Result for this assay is shown in FIG. 3.

### Example 5. Antibody Enhances NK-92 Cells Mediated Cytotoxicity to PLC/PRF/5 Cells

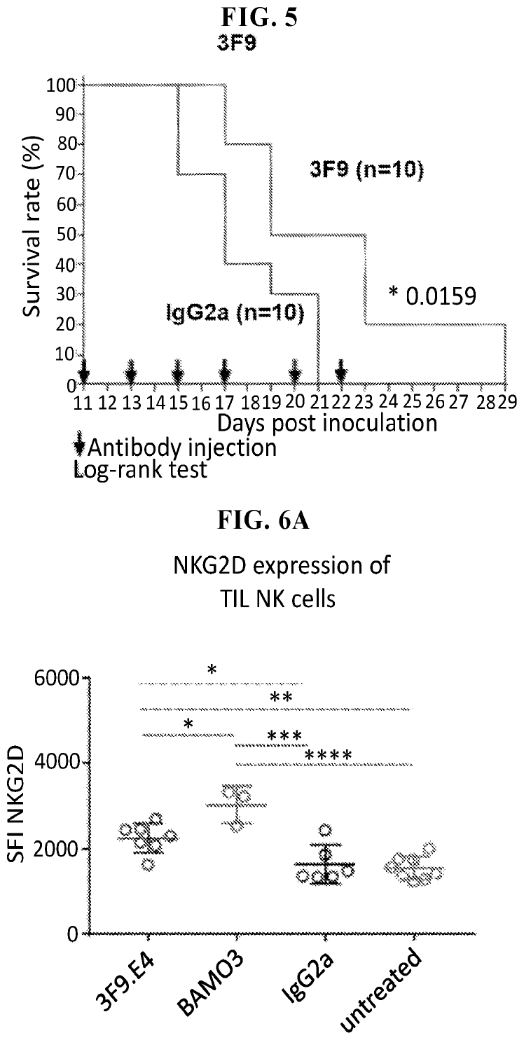

PLC/PRF/5 (Target) cells were suspended in RPMI-1640 with 10% FBS, and plated into 96-well flat bottom plates (Costar) at 6000 cells/well. Cells were then incubated with anti-MICA antibody (10 g/ml) that binds to MICA α3 domain for 24 hours before being labeled with calcein AM (1 μM) for 3 hours at 37° C., 5% CO₂. Wells were washed, and NK-92 cells (Effector) suspended in RPMI-1640 with 10% FBS were then added to wells at various Effector-to-Target (E:T) ratios as indicated and co-cultured with target cells for 4 hours. At the end of the cultures, the supernatant was removed, replaced with PBS, and the calcein AM signal was measured using a VICTOR Multilabel plate reader (Perkin Elmer). An isotype matched nonreactive immunoglobulin (R&D) antibody was used as a control. Results for this assay are shown in FIG. 4A-FIG. 4B.

### Example 6. Efficacy of 3F9.E4 in Tumor Therapy In Vivo (Survival) and NKG2D Expression by Tumor-Infiltrating Lymphocytes in Tumor-Bearing Mice

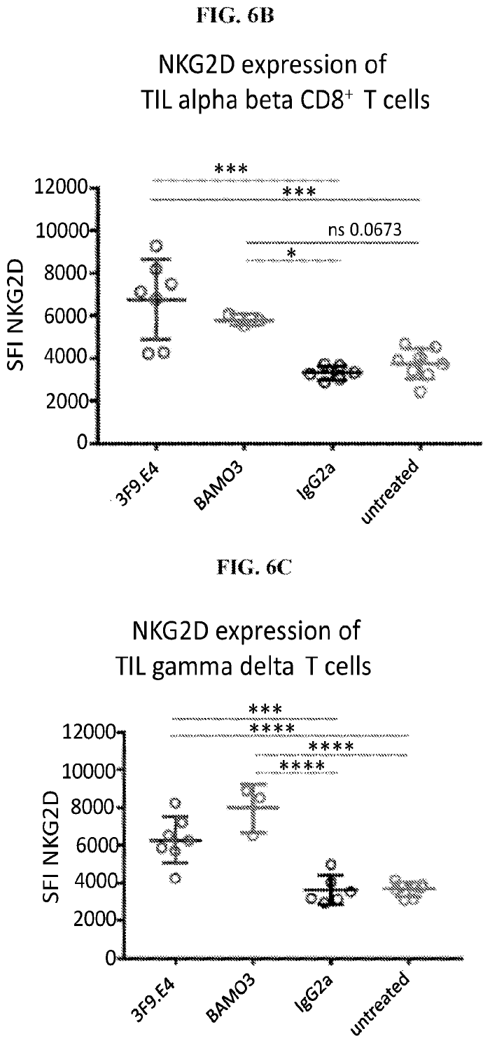

MICA transgenic mice (MICAgen) were inoculated with 10⁴ B16F10-MICA*01 cells (B16 transfectant) and, upon day 11, when tumors reached the desired threshold size, mice were treated with 3F9.E4 or isotype control (arrows indicate days of injection) (Log-rank test). Results are shown in FIG. 5.

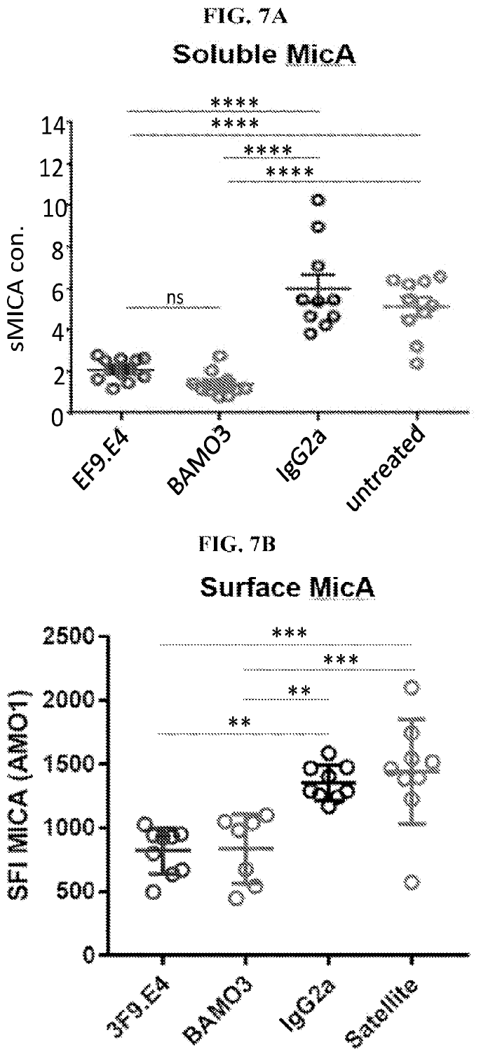

Mice were sacrificed when reaching a high tumor load and tumor-infiltrating lymphocytes analyzed for NKG2D expression (SFI) by NK cells, CD8 αβ T cells, and γδ T cells. NKG2D expression strength (SFI) of NKG2D-expressing cells are shown in FIG. 6A-FIG. 6C. Each symbol represents analyses of an individual mouse. (One-way ANOVA+Tukey's multiple comparisons test).

### Example 7. MICA Expression by B16 Transfectant Tumors Ex Vivo and Detection of Soluble MICA in Sera of Tumor-Bearing Mice

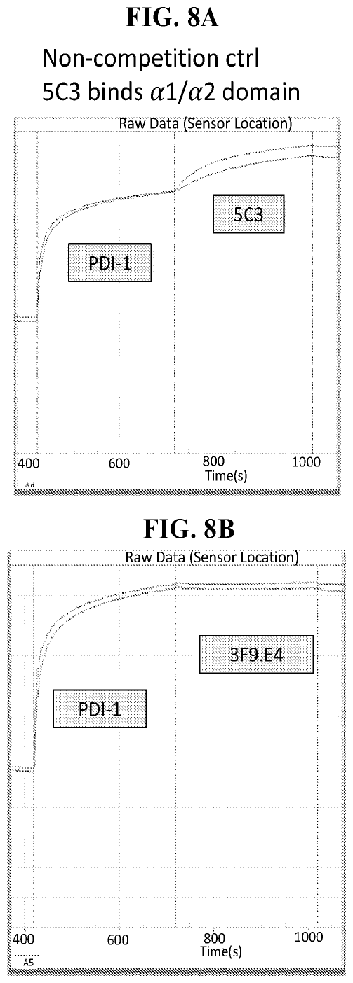

MICA transgenic mice (MICAgen) were inoculated with 10⁴ B16F10-MICA*01 cells (B16 transfectant) and, upon day 11, when tumors reached the desired threshold size, mice were treated with 3F9.E4 or BAMO3 or isotype control. Mice were sacrificed when reaching a high tumor load and isolated single tumor cells analyzed for MICA expression with mAb AMO1 staining (One-way ANOVA+Tukey's multiple comparisons test). AMO1 binds to the a1a2-domain of MICA (≠BAMO3 and 3F9). Each symbol represents analysis of an individual mouse. Results are shown in FIG. 7B. Sera was also analyzed for sMICA using AMO1/BAMO1 sandwich ELISA. Each symbol represents an individual mouse (Unpaired t-test). Results are shown in FIG. 7A.

### Example 8. Epitope Binning

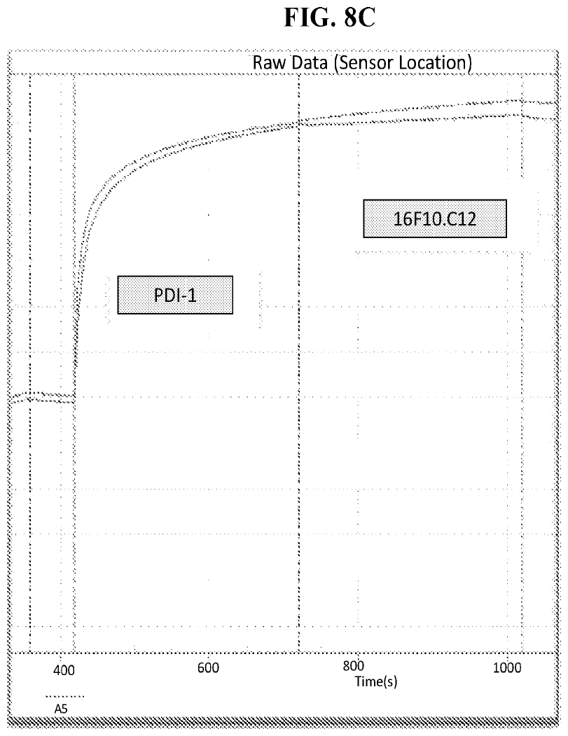

MICA *04-His protein (30 μg/ml) was immobilized onto Ni-NTA (FortrBio, #18-5101) biosensor tips for a biolayer interferometry instrument (Octet Red, ForteBio) for 300 s. The baseline signal was measured again for 60 s before biosensor tips were immersed into wells containing 1 mM or 0.5 mM of primary antibody for 300 s. Following this process, biosensors were immersed into wells containing 100 nM or 50 nM of a second mAb for 300 s. Percent binding of a second mAbs in the presence of the first mAb was determined by comparing the maximal signal of the second mAb after the first mAb was added to the maximum signal of the second mAb alone. mAbs were considered noncompeting if maximum binding of the second mAb was >66% of its uncompeted binding. A level between 33% and 66% of its uncompeted binding was considered intermediate competition, and <33% was considered competing. Results for this assay are shown in FIG. 8A-FIG. 8C.

### Example 9. Parameters Defining Binding of Fc Enhanced Antibodies to Human Fey Receptors

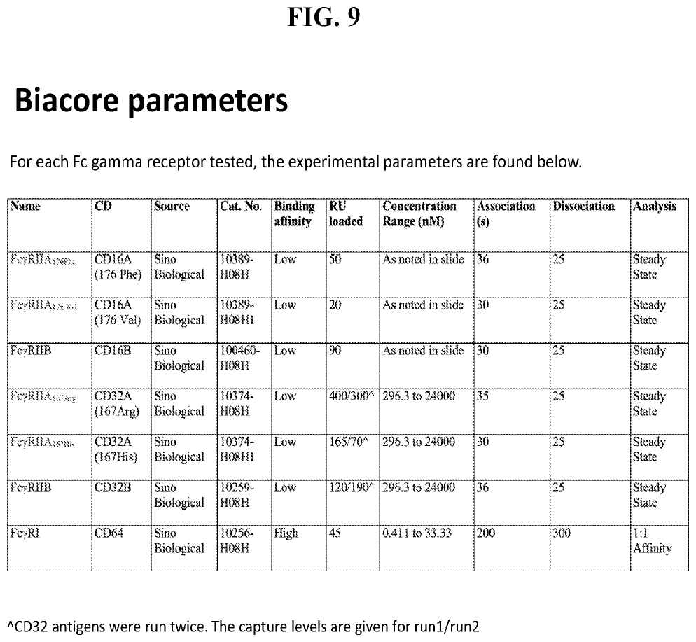

Binding of anti-MICA/B 3F9H1L3L antibody to human Fcγ Receptors was measured by Biacore, where the parameters for each Fcγ receptor tested for the Biacore analysis is shown in FIG. 9. Trastuzamb was used as an IgG1 control.

### Example 10. Human Fcγ Receptors Binding

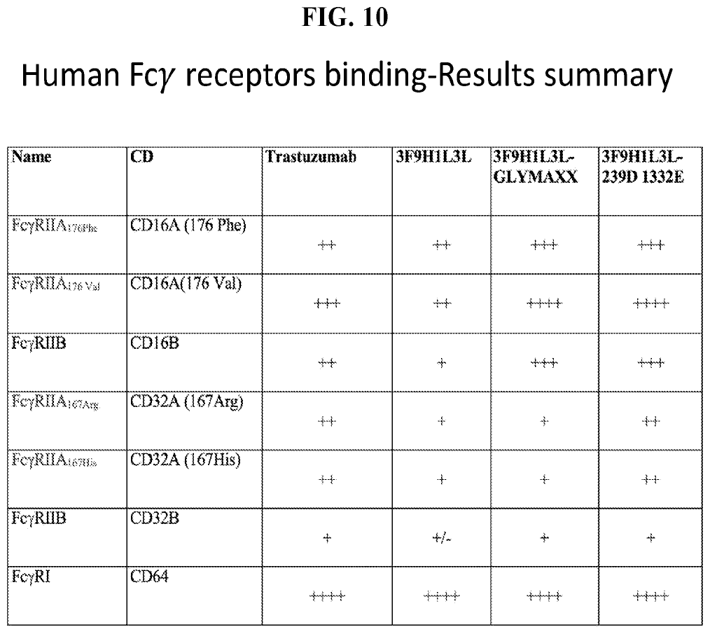

FIG. 10 shows the results of the Biacore analysis. Binding to FcγR1 is similar for wildtype 3F9H1L3L antibody, the afucosylated 3F9H1L3L (3F9H1L3L “GLYMAXX”) and 3F9H1L3L with the two heavy chain S239D and I332E mutations. Both afucosylated 3F9H1L3L and 3F9HIL3L with the two heavy chain S239D and I332E mutations have higher affinity to FcγRII and FcγRIII compared to wildtype antibody 3F9H1L3L.

### Example 11 Binding of Fe Enhanced Anti-MICA/B Antibodies to FcRn

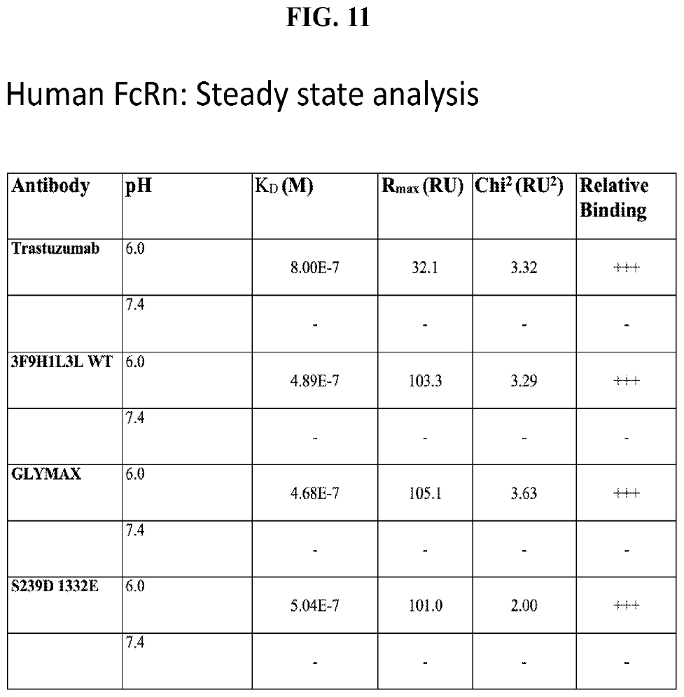

Binding of anti-MICA/B 3F9H1L3L antibody to human FcRn was measured by Biacore, FIG. 11 shows the results of the Biacore analysis. Binding to FcRn is similar for wildtype 3F9H1L3L antibody, the afucosylated 3F9H1L3L (3F9H1L3L “GLYMAXX”) and 3F9H1L3L with the two heavy chain S239D and I332E mutations at pH 6.0, and binding was pH dependent.

### Example 12: Fe Enhanced Anti-MICA/B Antibodies Bind to and Stabilize MICA/B on Cancer Cell Surface Similar to WT Anti-MICA/B Antibodies

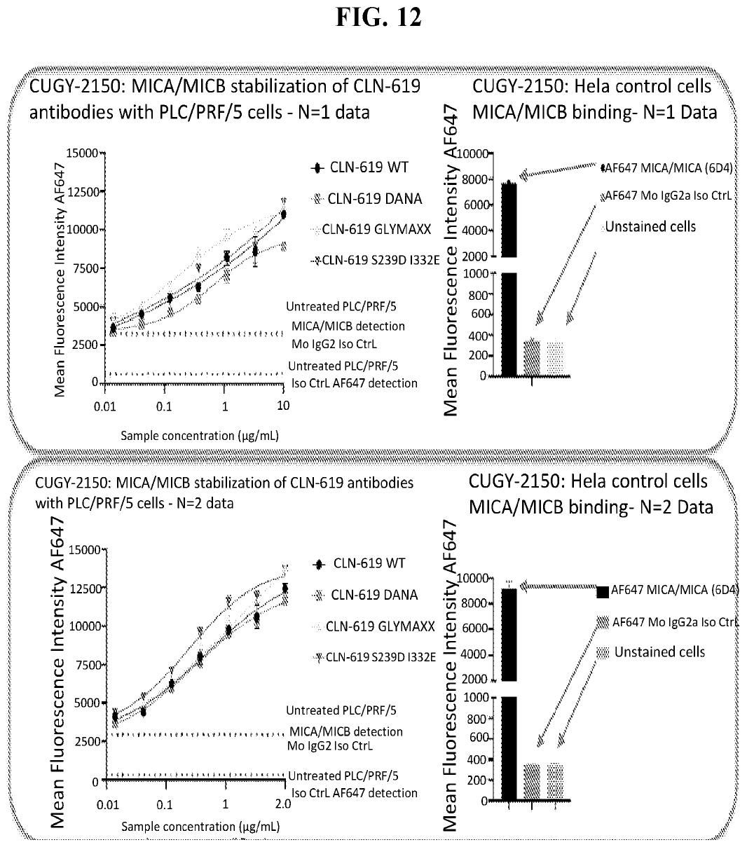

To confirm that anti-MICA/B antibodies and Fc enhanced anti-MICA/B antibodies stabilize MICA/B on the surface of cancer cells, MICA/B cell surface stabilization experiments were conducted in Hela cells and PLC/PRF/5 cells. Experiments were performed on Day 1 by seeding 20,000 cells per well of a 96-well assay plate. Cells were incubated overnight at 37° C./5% CO₂. On day two, cells were treated with test anti-MICA/B antibodies at 10 μg/mL starting concentration and 7-point 3-fold serial dilution. Cells were incubated for a further 24 h at 37° C./5% CO₂. On day three, cells were harvested in 96-well plates. Cells were washed and incubated with 1/50 diluted AlexaFluor 647 MICA/B in FACS buffer (PBS, 1% BSA, 0.1% NaN3) for 30 min on ice. Human IgG1 and Alexafluor 547 mouse IgG2aK isotype controls were included to confirm binding specificity. After 30 min, cells were washed and fixed. Fluorescence was then measured using Attune NxT focusing cytometer (ThermoFisher) and results are shown in FIG. 12. These results confirm that Fc enhanced anti-MICA/B antibodies bind to and stabilize MICA/B on cancer cell surface similar to WT anti-MICA/B antibodies.

### Example 13: Fc Enhanced Anti-MICA/B Antibodies Induce ADCC to a Higher Extent than Wild-Type Anti-MICA/B Antibodies

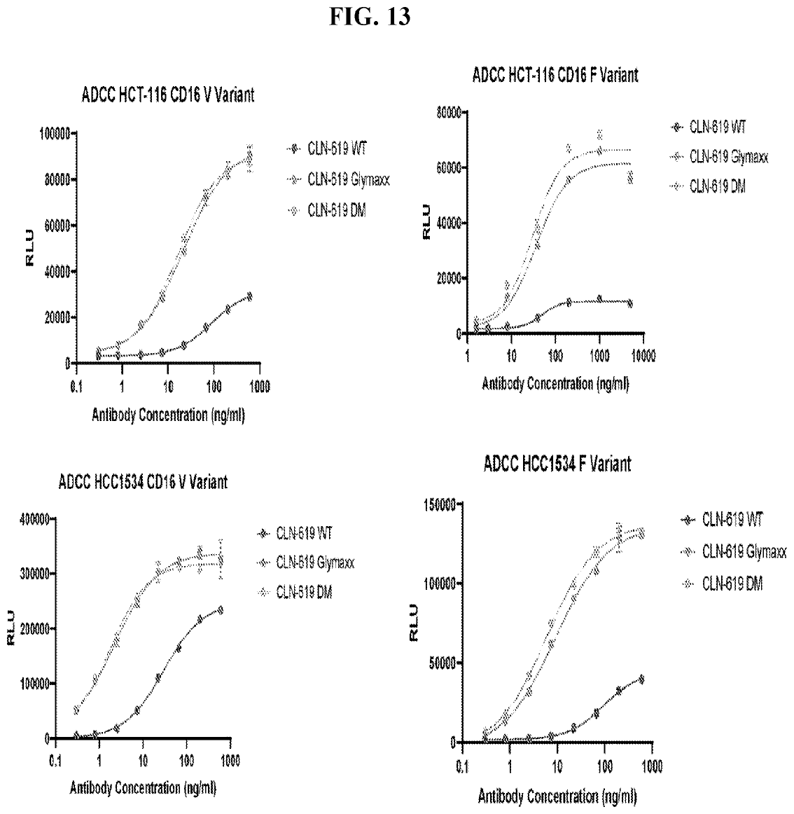

To confirm that Fc Enhanced anti-MICA/B antibodies can induce improved ADCC compared to wild-type or fucosylated MICA/B antibodies, the Promega ADCC Bioassay Complete Kit was used to measure ADCC of anti-MICA/B 3F9H1L3L antibody (CLN-619) using two target cell lines: HCC1534 cells, expressing high levels of MICA and HCT-116 cells, expressing low levels of MICA. Jurkat cells expressing high affinity variant (V) or low affinity variant (F) of FcγRIIIa were added to target cells with anti-MICA/B 3F9H1L3L wild type antibody (CLN-619), anti-MICA/B 3F9H1L3L afucosylated antibody (“GLYMAXX”), and anti-MICA/B 3F9H1L3L antibody with the two heavy chain S239D and I332E mutations (“CLN-619 DM”) and incubated for 6 h. After 6 h, luminescence was measure luminescence (FIG. 13). The anti-MICA/B antibodies with enhanced effector function lead to significantly more ADCC compared to the wild type anti-MICA/B antibody in both cell lines in all tested conditions when the cells were co-incubated with Jurkat cells expressing either the high or low affinity variants of FcγRIIa.

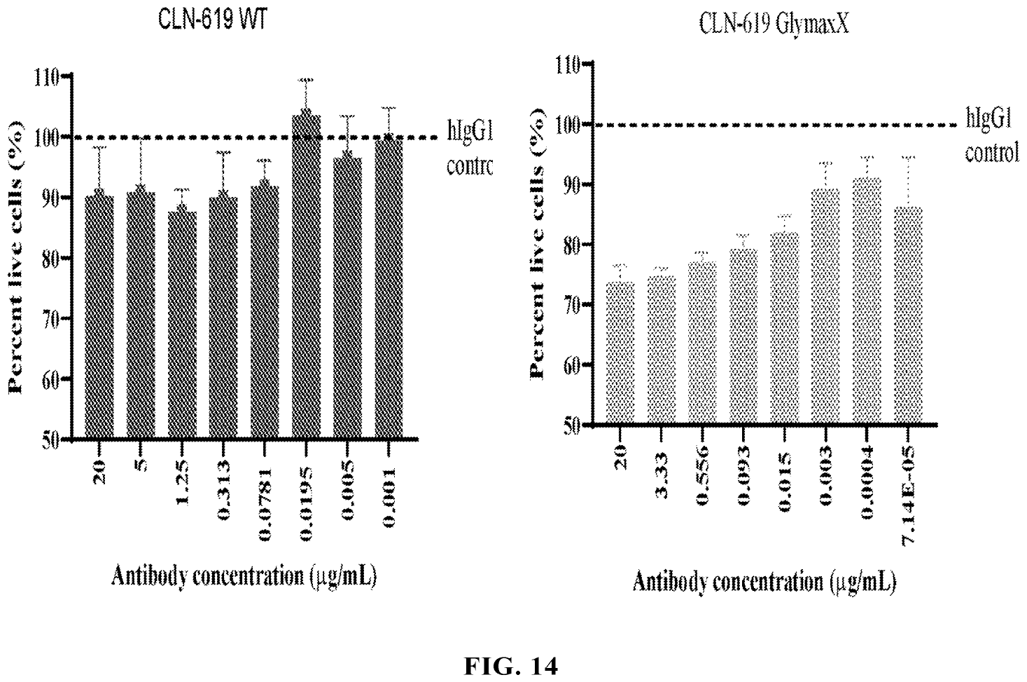

The ability of Fc Enhanced anti-MICA/B antibodies to induce cell killing was also determined. On day one, HCT-116 target cells were labeled with Cytolight® rapid red dye. Cells were seeded at 5000 cells/well in black, clear bottom 96-well plates. Target cells were incubated for 24 h at 37° C./5% CO₂. On day 3, NKG2D expressing effector cells were seeded onto target cells at an effector to target cell ratio of 40:1. Co-cultures were incubated in media containing a final assay concentration of 0.25 M Cytotox green dye to monitor cell killing. Fluorescence was measured using the Incucyte™ live cell imaging system every 30 min for 24 h to monitor cell killing. FIG. 14 shows HCT-116 cells incubated with afucosylated anti-MICA/B 3F9HIL3L antibody (CLN-619 GLYMAXX) either with or without addition of NKG2D effector cells caused a dose dependent decrease in the percentage of cells. Wild-type anti-MICA/B 3F9H1L3L antibody (CLN-619 WT) also caused a reduction in the percentage of HCT-116 cells with incubation of NKG2D effector cells; but the reduction in cells was less pronounced than afucosylated anti-MICA/B 3F9H1L3L antibody.

These results show that anti-MICA/B antibodies with enhanced Fc function increases the ability of the anti-MICA/B antibody to induce ADCC and cancer cell death.

### Example 14: Efficacy Measured by Survival Analysis

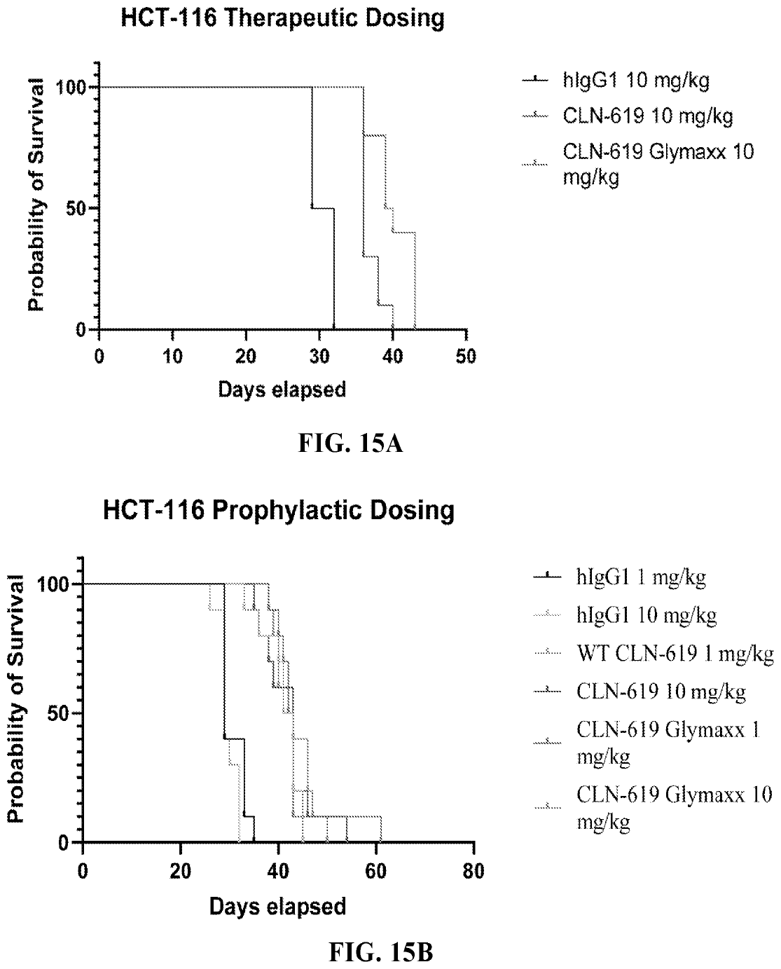

The efficacy of CLN-619 WT was compared to CLN-619 Glymaxx (afucosylated) head-to-head as single agents administered as a prophylactic treatment starting on Day 1 or as a therapeutic treatment starting on Day 4 in the HCT-116-Luc human colon tumor xenograft model. Female Balb/c SCID mice were inoculated intraperitoneally (IP) to model disseminated disease. Treatment was given twice weekly IP. Bioluminescent imaging (BLI) was preformed to monitor progression of the disseminated disease. Significant endpoints were the assessment of treatment effects on tumor growth inhibition as measured by BLI (not shown) and time to peri-morbidity endpoint (survival) is shown in FIG. 15A. The study ended on Day 61, when the final mouse reached a peri-moribund state. Survival analysis was done by log rank test utilizing Graphpad software. All CLN-619 WT and CLN-619 Glymaxx treated groups, both prophylactic and therapeutic, resulted in a significant increase in survival when compared to their respective higG1 control group as shown in FIG. 15B. In the prophylactic setting, CLN-619 WT and CLN-619 Glymaxx are not significantly different from each other. In the therapeutic setting, CLN-619 Glymaxx dosed at 10 mg/kg was statistically superior to CLN-619 dosed at 10 mg/kg (P=0.003).

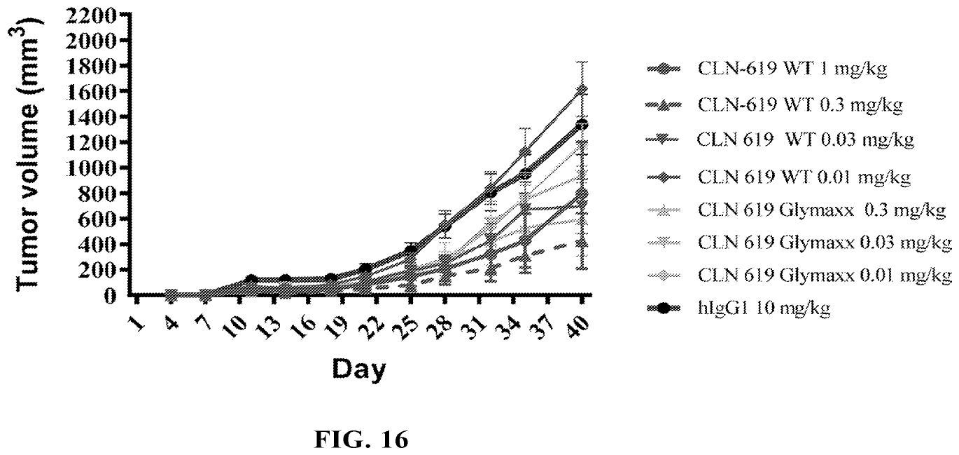

CLN-619 WT and CLN-619 Glymaxx were compared head-to-head in an in vivo efficacy study was performed in the HCC1534 lung tumor xenograft model implanted in Balb/c SCID mice. As shown in FIG. 16, treatment with CLN-619 WT, CLN-619 Glymaxx or hIgG1 control began on Day 1, the day of tumor cell implantation. Treatment was administered two times a week for four weeks by IP injection. Doses ranged from 0.01 to 1 mg/kg of CLN-619 test articles and 1 mg/kg of hIgG1 control antibody. The study was terminated on Day 40, and statistical analyses were calculated based on Day 40. Statistical analysis accounted for multiple comparisons, using one-way ANOVA and Tukey's multiple comparisons and analysis was carried out in graphpad. No differences in efficacy were observed between WT vs. Fc-enhanced at any dose in the HCC1534 cell line.

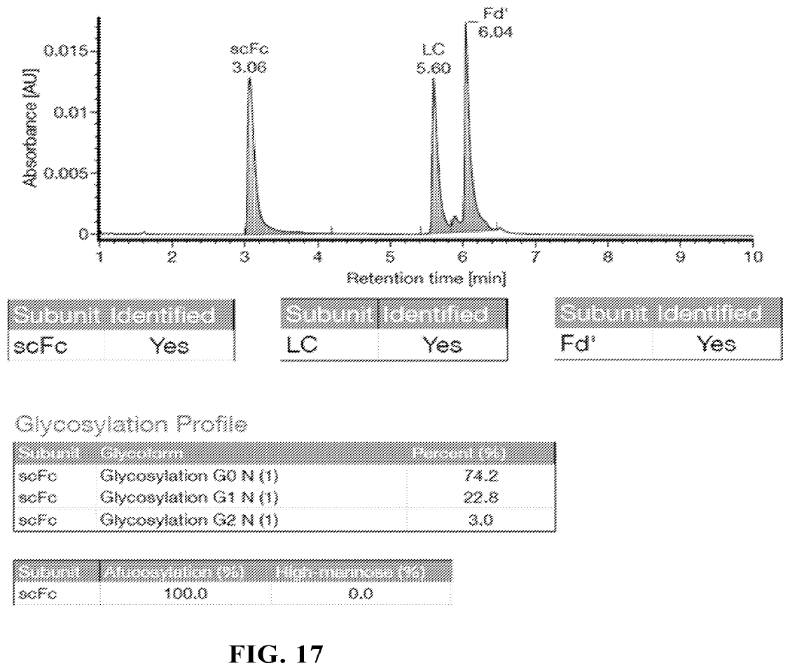

The working principle of the GlymaxX® Technology is in the absence of fucose, cells are unable to synthesize GDP-fucose via the salvage pathway. The de novo pathway, the dominant source of activated GDP-L-Fucose, is efficiently blocked by enzymatic conversion of the intermediate GDP-4-keto-6-deoxymannose into GDP-D-Rhamnose, a dead end product that typically does not occur in vertebrate cells. Production of non-fucosylated antibodies by co-expression of heterologous GDP-6-deoxy-D-lyxo-4-hexulose reductase. Briefly, the antibody was digested with protease to yield three subunits ((scFc, Light chain and Fd′) and subunits were analyzed by reverse-phase LC-MS. Afucosylation was measured using mass spectrometry analysis. Representative lot of CLN-619 Glymaxx (afucosylated antibody) that was generated using GlymaxX® Technology is shown in FIG. 17.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A monoclonal antibody or an antigen-binding fragment thereof, comprising:
(i) a light chain comprising an amino acid sequence at least 80% identical to the amino acid sequence set forth as SEQ ID NO: 21, SEQ ID NO: 23, or SEQ ID NO: 24, and
a heavy chain comprising an amino acid sequence at least 80% identical to the amino acid sequence set forth as SEQ ID NO: 22, SEQ ID NO: 25, or SEQ ID NO: 26; or
(ii) a light chain variable domain (VL) comprising an amino acid sequence at least 80% identical to the amino acid sequence set forth as SEQ ID NO: 7, SEQ ID NO: 15, or SEQ ID NO: 19; and
a heavy chain variable domain (VH) comprising an amino acid sequence at least 80%, identical to the amino acid sequence set forth as SEQ ID NO: 8, SEQ ID NO: 16, or SEQ ID NO: 20; and
wherein the antibody or the antigen-binding fragment thereof:
a) comprises one or more amino acid modifications to enhance effector function compared to the unmodified monoclonal antibody or antigen-binding fragment thereof; and/or
b) lacks fucosylation or comprises reduced fucosylation compared to an amount of fucose normally detected for a similar antibody produced by a mammalian expression system.

2-10. (canceled)

11. The monoclonal antibody or antigen-binding fragment thereof of claim 1, wherein the monoclonal antibody or antigen-binding fragment thereof specifically binds to a MICA protein, a MICB protein, or both MICA and MICB protein.

12-14. (canceled)

15. The monoclonal antibody or antigen-binding fragment thereof of claim 1, wherein the monoclonal antibody or antigen-binding fragment thereof is selected from a whole immunoglobulin, an scFv, a Fab, a F(ab′)2, or a disulfide linked Fv.

16. The monoclonal antibody of claim 1, wherein the monoclonal antibody or antigen-binding fragment thereof is an IgG or IgM.

17. The monoclonal antibody of claim 1, wherein the monoclonal antibody or antigen-binding fragment thereof is humanized or chimeric.

18-35. (canceled)

36. A monoclonal antibody or an antigen-binding fragment thereof, wherein the monoclonal antibody or antigen-binding fragment thereof comprises a light chain complementarity determining region 1 (LCDR1) comprising an amino acid sequence at least 80% identical to one of SEQ ID NO: 1, SEQ ID NO: 9, or SEQ ID NO: 17, a light chain complementarity determining region 2 (LCDR2) comprising an amino acid sequence at least 80% identical to one of SEQ ID NOS: SEQ ID NO: 2, or SEQ ID NO: 10, a light chain complementarity determining region 3 (LCDR3) comprising an amino acid sequence at least 80% identical to one of SEQ ID NO: 3, or SEQ ID NO: 11, a heavy chain complementarity determining region 1 (HCDR1) comprising an amino acid sequence at least 80% identical to one of SEQ ID NO: 4, or SEQ ID NO: 12, a heavy chain complementarity determining region 2 (HCDR2) comprising having an amino acid sequence at least 80% identical to one of SEQ ID NO: 5, SEQ ID NO: 13, or SEQ ID NO: 18, and a heavy chain complementarity determining region 3 (HCDR3) comprising having an amino acid sequence at least 80% identical to one of SEQ ID NO: 6, or SEQ ID NO: 14.

37-50. (canceled)

51. The monoclonal antibody or antigen-binding fragment thereof of claim 36, wherein the monoclonal antibody or antigen-binding fragment thereof specifically binds to a MICA protein, a MICB protein, or both MICA and MICB protein.

52-54. (canceled)

55. The monoclonal antibody or antigen-binding fragment thereof of claim 36, wherein the monoclonal antibody or antigen-binding fragment thereof is selected from a whole immunoglobulin, an scFv, a Fab, a F(ab′)2, or a disulfide linked Fv.

56. The monoclonal antibody or antigen-binding fragment thereof of claim 36, wherein the monoclonal antibody or antigen-binding fragment thereof is an IgG or IgM.

57. The monoclonal antibody or antigen-binding fragment thereof of claim 36, wherein the monoclonal antibody or antigen-binding fragment thereof is humanized or chimeric.

58. The monoclonal antibody or an antigen-binding fragment thereof, of claim 36 wherein the antibody or an antigen-binding fragment thereof:
a) comprises one or more amino acid modifications to enhance effector function compared to the unmodified monoclonal antibody or antigen-binding fragment thereof; and/or
b) lacks fucosylation or comprises reduced fucosylation compared to an amount of fucose normally detected for a similar antibody produced by a mammalian expression system.

59-148. (canceled)

149. The monoclonal antibody or an antigen-binding fragment thereof of claim 36, wherein the monoclonal antibody or an antigen-binding fragment thereof comprises a mutation in:
(i) the Fc region at amino acid 239;
(ii) the Fc region at amino acid 332;
(iii) the Fc region S239D:
(iv) the Fc region I332D; and/or
(v) one or more amino acid modifications of Table B and/or Table C.

150-156. (canceled)

157. The monoclonal antibody or an antigen-binding fragment thereof of claim 36, wherein the monoclonal antibody or an antigen-binding fragment thereof comprises less than 95% of an amount of fucose normally detected for a similar antibody produced by a mammalian expression system.

158-188. (canceled)

189. The monoclonal antibody or an antigen-binding fragment thereof of claim 1, wherein the monoclonal antibody or an antigen-binding fragment thereof comprises a mutation in:
(i) the Fc region at amino acid 239;
(ii) the Fc region at amino acid 332;
(iii) the Fc region S239D;
(iv) the Fc region I332D; and/or
(v) one or more amino acid modifications of Table B and/or Table C.

190. The monoclonal antibody or an antigen-binding fragment thereof of claim 1, wherein the monoclonal antibody or an antigen-binding fragment thereof comprises less than 95% of an amount of fucose normally detected for a similar antibody produced by a mammalian expression system.


SEQUENCE LISTING (35 sequences)

SEQ ID NO: 1 - protein, 16 aa, synthetic construct
  REGION 1..16: note = 3F9.E4 Light Chain CDR1
RSSKSLLHSNGNTYLY

SEQ ID NO: 2 - protein, 7 aa, synthetic construct
  REGION 1..7: note = 3F9.E4 Light Chain CDR2
  REGION 1..7: note = 3F9H1L3L Light Chain CDR2
RMSNLAS

SEQ ID NO: 3 - protein, 9 aa, synthetic construct
  REGION 1..9: note = 3F9.E4 Light Chain CDR3
  REGION 1..9: note = 3F9H1L3L Light Chain CDR3
MQHLEYPFT

SEQ ID NO: 4 - protein, 10 aa, synthetic construct
  REGION 1..10: note = 3F9.E4 Heavy Chain CDR1
  REGION 1..10: note = 3F9H1L3L Heavy Chain CDR1
GFTFSNYAMS

SEQ ID NO: 5 - protein, 17 aa, synthetic construct
  REGION 1..17: note = 3F9.E4 Heavy Chain CDR2
YISPGGDYIYYADTVKG

SEQ ID NO: 6 - protein, 12 aa, synthetic construct
  REGION 1..12: note = 3F9.E4 Heavy Chain CDR3
  REGION 1..12: note = 3F9H1L3L Heavy Chain CDR3
DRRHYGSYAMDY

SEQ ID NO: 7 - protein, 112 aa, synthetic construct
  REGION 1..112: note = 3F9.E4 Light Chain Variable domain
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 8 - protein, 121 aa, synthetic construct
  REGION 1..121: note = 3F9.E4 Heavy Chain Variable domain
EVQLQESGEGLVKPGGSLKLSCAASGFTFSNYAMSWVRQTPEKRLEWVAYISPGGDYIYYADTVKGRFTISRDNARNTLYLQMSSLKSEDTAMYYCTTDRRHYGSYAMDYWGQGISVTVSS

SEQ ID NO: 9 - protein, 12 aa, synthetic construct
  REGION 1..12: note = 16F10.C12 Light Chain CDR1
TASSSVSSNYLH

SEQ ID NO: 10 - protein, 7 aa, synthetic construct
  REGION 1..7: note = 16F10.C12 Light Chain CDR2
TTSNLAS

SEQ ID NO: 11 - protein, 9 aa, synthetic construct
  REGION 1..9: note = 16F10.C12 Light Chain CDR3
HQFHRSPFT

SEQ ID NO: 12 - protein, 10 aa, synthetic construct
  REGION 1..10: note = 16F10.C12 Heavy Chain CDR1
GFSLTAFGVN

SEQ ID NO: 13 - protein, 16 aa, synthetic construct
  REGION 1..16: note = 16F10.C12 Heavy Chain CDR2
MIWGDGNTDYNSTLRS

SEQ ID NO: 14 - protein, 12 aa, synthetic construct
  REGION 1..12: note = 16F10.C12 Heavy Chain CDR3
ETYYGNYAGLGY

SEQ ID NO: 15 - protein, 108 aa, synthetic construct
  REGION 1..108: note = 16F10.C12 Light Chain Variable domain
QIVLTQSPAIMSASIGERVTMTCTASSSVSSNYLHWYQQKPRSSPKLWIYTTSNLASGVPTRFSGSGSGTSYSLTISSMEAEDAATYYCHQFHRSPFTFGSGTKLEIK

SEQ ID NO: 16 - protein, 120 aa, synthetic construct
  REGION 1..120: note = 16F10.C12 Heavy Chain Variable domain
EVQLQESGPGLVAPSQSLSITCTVSGFSLTAFGVNWVRQPPGKGLEWLGMIWGDGNTDYNSTLRSRLSISKDNSKSQVFLKLNSLQTDDTARYFCARETYYGNYAGLGYWGQGTLVTVSA

SEQ ID NO: 17 - protein, 16 aa, synthetic construct
  REGION 1..16: note = 3F9H1L3L Light Chain CDR1
RSSKSLLHSNLNTYLY

SEQ ID NO: 18 - protein, 17 aa, synthetic construct
  REGION 1..17
  REGION 1..17: note = 3F9H1L3L Heavy Chain CDR2
YISPGGDYIYYADSVKG

SEQ ID NO: 19 - protein, 113 aa, synthetic construct
  REGION 1..113: note = 3F9H1L3L Light Chain Variable domain
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIKR

SEQ ID NO: 20 - protein, 121 aa, synthetic construct
  REGION 1..121: note = 3F9H1L3L Heavy Chain Variable domain
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 21 - protein, 219 aa, synthetic construct
  REGION 1..219: note = 3F9H1L3L Light Chain
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 450 aa, synthetic construct
  REGION 1..450: note = 3F9H1L3L Heavy Chain
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 234 aa, synthetic construct
  REGION 1..234: note = PDI-1 humanized LC5 Light Chain
METDTLLLWVLLLWVPGSTGDIQMTQSPSTLSASVGDRVTITCSASQGISNYLNWYQQKPGKAPKLLIQYTSLLHSGVPSRFSGSGSGTEYTLTISSLQPDDFATYFCQQYSKFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 234 aa, synthetic construct
  REGION 1..234: note = PDI-1 humanized LC4 Light Chain
METDTLLLWVLLLWVPGSTGDIQMTQSPSSLSASVGDRVTITCSASQGISNYLNWYQQKPGKAPKLLIQYTSLLHSGVPSRFSGSGSGTDYTLTISSLQPEDFATYFCQQYSKFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 471 aa, synthetic construct
  REGION 1..471: note = PDI-1 humanized HC4 Heavy Chain
MDPKGSLSWRILLFLSLAFELSYGQIQLVQSGSELKKPGASVKVSCKAFGYTFTNYGMNWVKQAPGQGLKWMGWINTYTGEPTYAQGFTGRFVFSLETSVSTAYLQISSLKAEDTAVYFCARNYGNYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 471 aa, synthetic construct
  REGION 1..471: note = PDI-1 humanized HC6 Heavy Chain
MDPKGSLSWRILLFLSLAFELSYGQIQLVQSGAEVKKPGASVKVSCKAFGYTFTNYGMNWVKQAPGQGLKWMGWINTYTGEPTYADDFKGRVTFTLETSISTAYMELSRLRSDDTAVYFCARNYGNYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 108 aa, synthetic construct
  REGION 1..108: note = PDI-1 Light Chain Variable domain
DIQMTQTTSSLSASLGDRVTISCSASQGISNYLNWYQQKPDGTLKLLIQYTSLLHSGVPSRFSGSGSGTEYSLTISNLEPEDIATYFCQQYSKFPRTFGGGTKLEIKR

SEQ ID NO: 28 - protein, 118 aa, synthetic construct
  REGION 1..118: note = PDI-1 Heavy Chain Variable domain
QIQLVQSGPELKKSGETVKISCKAFGYTFTNYGMNWVKQAPGKGLKWMGWINTYTGEPTYADDFKGRFAFSLETSASTAYLQINHLKNEDTATYFCARNYGNYLFDYWGQGTTLTVSS

SEQ ID NO: 29 - protein, 450 aa, synthetic construct
  REGION 1..450: note = 3F9H1L3L Heavy Chain with Exemplary Enhanced Fc
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 11 aa, synthetic construct
SASQGISNYLN

SEQ ID NO: 31 - protein, 7 aa, synthetic construct
TSLLHSG

SEQ ID NO: 32 - protein, 9 aa, synthetic construct
QQYSKFPRT

SEQ ID NO: 33 - protein, 10 aa, synthetic construct
GYTFTNYGMN

SEQ ID NO: 34 - protein, 16 aa, synthetic construct
INTYTGEPTYADDFKG

SEQ ID NO: 35 - protein, 9 aa, synthetic construct
NYGNYLFDY